SEQUENCE LISTING (139 sequences)

SEQ ID NO: 1 - DNA, 20 bp, Artificial Sequence
  synthetic tracrRNA/crRNA
ttgcgcacggttatgtggac

SEQ ID NO: 2 - DNA, 25 bp, Artificial Sequence
  oJML0053 primer
tttccataggctccgcccccctgac

SEQ ID NO: 3 - DNA, 30 bp, Artificial Sequence
  oJML0056 primer
gagagcaatcccgcaatcttcagtggtgtg

SEQ ID NO: 4 - DNA, 30 bp, Artificial Sequence
  oJML0057 primer
cacaccactgaagattgcgggattgctctc

SEQ ID NO: 5 - DNA, 60 bp, Artificial Sequence
  oJML0058 primer
tgattactattaataactagtcaataatcaatgtcaacgcggtatttcacaccgcataga

SEQ ID NO: 6 - DNA, 40 bp, Artificial Sequence
  oJML0067 primer
gcaatgataccgcgagatccacgctcaccggctccagatt

SEQ ID NO: 7 - DNA, 40 bp, Artificial Sequence
  oJML0068 primer
aatctggagccggtgagcgtggatctcgcggtatcattgc

SEQ ID NO: 8 - DNA, 55 bp, Artificial Sequence
  oJML0069 primer
ttatttttatagcacgtgatgaaaaggaccaacacagtcctttcccgcaattttc

SEQ ID NO: 9 - DNA, 60 bp, Artificial Sequence
  oJML0070 primer
aaaaagaaaattgcgggaaaggactgtgttggtccttttcatcacgtgctataaaaataa

SEQ ID NO: 10 - DNA, 38 bp, Artificial Sequence
  oJML0126 primer
gtggtggaagactcgagcatgaccgagtacaagcccac

SEQ ID NO: 11 - DNA, 38 bp, Artificial Sequence
  oJML0129 primer
gttggtggcgccgctgccgttagcctcccccatctccc

SEQ ID NO: 12 - DNA, 60 bp, Artificial Sequence
  oJML0130 primer
tgaagcaggccggcgacgtggaggagaaccccggccccatggccaatttactgaccgtac

SEQ ID NO: 13 - DNA, 31 bp, Artificial Sequence
  oJML0131 primer
ggagcgccagacgaggccaatcatcaggatc

SEQ ID NO: 14 - DNA, 31 bp, Artificial Sequence
  oJML0132 primer
gatcctgatgattggcctcgtctggcgctcc

SEQ ID NO: 15 - DNA, 60 bp, Artificial Sequence
  oJML0133 primer
tgcgatgaagtagagcccgcagtggccaagtggctttggtccgtttcctccacggatgcc

SEQ ID NO: 16 - DNA, 60 bp, Artificial Sequence
  oJML0134 primer
gaaaccctctgcctcccccgtgatgtaatacttttgcaaggaatgcgatgaagtagagcc

SEQ ID NO: 17 - DNA, 60 bp, Artificial Sequence
  oJML0135 primer
gggaggcagagggtttccctgccacagcttgatgaagatgaggccaaccttctatcagag

SEQ ID NO: 18 - DNA, 49 bp, Artificial Sequence
  oJML0136 primer
gtggtggaagactctgaatgtctcaaaaaacaaacgaacaaaaaaccag

SEQ ID NO: 19 - DNA, 40 bp, Artificial Sequence
  oJML0137 primer
catgacccgcaagcccggtgccatgcccacgctactgcgg

SEQ ID NO: 20 - DNA, 60 bp, Artificial Sequence
  oJML0138 primer
ggggttctcctccacgtcgccggcctgcttcagcaggctgaagttggtggcgccgctgcc

SEQ ID NO: 21 - DNA, 60 bp, Artificial Sequence
  oJML0139 primer
ggcgccaccaacttcagcctgctgaagcaggccggcgacgtggaggagaaccccggcccc

SEQ ID NO: 22 - DNA, 40 bp, Artificial Sequence
  oJML0144 primer
aaacccgcagtagcgtgggcatggcaccgggcttgcgggt

SEQ ID NO: 23 - DNA, 37 bp, Artificial Sequence
  oJML0145 primer
gtggtgcgtctcttcggggctccggtgcccgtcagtg

SEQ ID NO: 24 - DNA, 38 bp, Artificial Sequence
  oJML0146 primer
caccaccgtctcggctctcacgacacctgaaatggaag

SEQ ID NO: 25 - DNA, 34 bp, Artificial Sequence
  oRB_036 primer
atcggcggccgctggaattataacttcgtatagc

SEQ ID NO: 26 - DNA, 29 bp, Artificial Sequence
  oRB_037 primer
ggccatcgatttacttgtacagctcgtcc

SEQ ID NO: 27 - DNA, 70 bp, Artificial Sequence
  oRB_061 primer
gattattagggataacagggtaatataacttcgtataggatactttatacgaagttatggctccggtgcc

SEQ ID NO: 28 - DNA, 39 bp, Artificial Sequence
  oRB_062 primer
gcatggacgagctgtacaagggcagtggagagggcagag

SEQ ID NO: 29 - DNA, 33 bp, Artificial Sequence
  oRB_063 primer
cctctgccctctccactgcccttgtacagctcg

SEQ ID NO: 30 - DNA, 78 bp, Artificial Sequence
  oRB_064 primer
cctaaaattaccctgttatccctaataacttcgtataatgtatgctatacgaagttattccccagcatgcctgctatt

SEQ ID NO: 31 - DNA, 60 bp, Artificial Sequence
  Left HA forward primer
tgtgtggtctcaccctttcatacccatgtaaggttgaggtgaagagactgaggtccagag

SEQ ID NO: 32 - DNA, 35 bp, Artificial Sequence
  Left HA reverse primer
gtggtgggtctcacgttggcgcgcgttgcttcatg

SEQ ID NO: 33 - DNA, 40 bp, Artificial Sequence
  Right HA forward primer
gtggtgggtctcgtatgttgaggtagatgttaccacatgt

SEQ ID NO: 34 - DNA, 60 bp, Artificial Sequence
  Right HA reverse primer
gtggtgggtctcaggatatgaagcaacgcgcgccggtaggtctggacctgcacttcttca

SEQ ID NO: 35 - DNA, 38 bp, Artificial Sequence
  Left HA forward primer
gtgtgtggtctcacccttgaagagactgaggtccagag

SEQ ID NO: 36 - DNA, 35 bp, Artificial Sequence; identical to SEQ ID NO: 32
  Left HA reverse primer

SEQ ID NO: 37 - DNA, 40 bp, Artificial Sequence; identical to SEQ ID NO: 33
  Right HA forward primer

SEQ ID NO: 38 - DNA, 37 bp, Artificial Sequence
  Right HA reverse primer
gtggtgggtctcaggattctggacctgcacttcttca

SEQ ID NO: 39 - DNA, 41 bp, Artificial Sequence
  Left HA forward primer
gtgtggtctcaccctgtacaaaactacagagcagttaagtg

SEQ ID NO: 40 - DNA, 35 bp, Artificial Sequence; identical to SEQ ID NO: 32
  Left HA reverse primer

SEQ ID NO: 41 - DNA, 40 bp, Artificial Sequence; identical to SEQ ID NO: 33
  Right HA forward primer

SEQ ID NO: 42 - DNA, 36 bp, Artificial Sequence
  Right HA reverse primer
ggtgggtctcaggatgttatacgacgccaaactgcc

SEQ ID NO: 43 - DNA, 35 bp, Artificial Sequence
  Left HA forward primer
gtgtggtctcaccctaaggtcttgggaatgggacg

SEQ ID NO: 44 - DNA, 35 bp, Artificial Sequence; identical to SEQ ID NO: 32
  Left HA reverse primer

SEQ ID NO: 45 - DNA, 40 bp, Artificial Sequence; identical to SEQ ID NO: 33
  Right HA forward primer

SEQ ID NO: 46 - DNA, 34 bp, Artificial Sequence
  Right HA reverse primer
ggtgggtctcaggatgagggtagccaagtggacc

SEQ ID NO: 47 - DNA, 58 bp, Artificial Sequence
  Left HA forward primer
gtgtggtctcaccctcaagtctgaagtagttcaggagggtttgaggccaggaagggat

SEQ ID NO: 48 - DNA, 37 bp, Artificial Sequence
  Left HA reverse primer
gtggtgggtctcacgttgaactacttcagacttgggc

SEQ ID NO: 49 - DNA, 43 bp, Artificial Sequence
  Right HA forward primer
gtggtgggtctcgtatggttcggggacggtgttaatattcttc

SEQ ID NO: 50 - DNA, 58 bp, Artificial Sequence
  Right HA reverse primer
ggtgggtctcaggatgagctgcaaaggctcccgttaggaatgaatgcggatgccttgc

SEQ ID NO: 51 - DNA, 58 bp, Artificial Sequence
  Left HA forward primer
ggtgggtctcaccctcatcgtgcccatagcaatgatgggcccaggtgaagagtcaacc

SEQ ID NO: 52 - DNA, 35 bp, Artificial Sequence
  Left HA reverse primer
ggtgggtctcacgttttgctatgggcacgatgctg

SEQ ID NO: 53 - DNA, 35 bp, Artificial Sequence
  Right HA forward primer
ggtgggtctcgtatgagagctgggataatctcttt

SEQ ID NO: 54 - DNA, 59 bp, Artificial Sequence
  Right HA reverse primer
ggtgggtctcaggatgagattatcccagctctgggaggagctattttagaaggactccc

SEQ ID NO: 55 - DNA, 20 bp, Artificial Sequence
  hPIGA-g1
gttatactttggccagcatg

SEQ ID NO: 56 - DNA, 20 bp, Artificial Sequence
  hPIGA-g2
aacatctagccacatccatt

SEQ ID NO: 57 - DNA, 20 bp, Artificial Sequence
  HPRT1-g1
atgaagcaacgcgcgccggt

SEQ ID NO: 58 - DNA, 20 bp, Artificial Sequence
  HPRT1-g2
ttcatacccatgtaaggttg

SEQ ID NO: 59 - DNA, 20 bp, Artificial Sequence
  mPiga-g1
ggcatgctttgtggtcgttc

SEQ ID NO: 60 - DNA, 20 bp, Artificial Sequence
  mPiga-g2
cccgcgggcagcctatataa

SEQ ID NO: 61 - DNA, 20 bp, Artificial Sequence
  Sox2-g1
caagtctgaagtagttcagg

SEQ ID NO: 62 - DNA, 20 bp, Artificial Sequence
  Sox2-g2
gagctgcaaaggctcccgtt

SEQ ID NO: 63 - DNA, 20 bp, Artificial Sequence
  Sox2-g3
cattggcagtgttgtatagg

SEQ ID NO: 64 - DNA, 20 bp, Artificial Sequence
  Igf2/H19-g1
catcgtgcccatagcaatga

SEQ ID NO: 65 - DNA, 20 bp, Artificial Sequence
  Igf2/H19-g2
gagattatcccagctctggg

SEQ ID NO: 66 - DNA, 22 bp, Artificial Sequence
  forward primer for LP-TK at HPRT1 L Jx
caggatatttctctgttgccca

SEQ ID NO: 67 - DNA, 18 bp, Artificial Sequence
  reverse primer for LP-TK at HPRT1 L Jx
gggactgtgggcgatgtg

SEQ ID NO: 68 - DNA, 19 bp, Artificial Sequence
  forward primer for LP-TK at HPRT1 R Jx
acccacagcttctcaacgg

SEQ ID NO: 69 - DNA, 20 bp, Artificial Sequence
  reverse primer for LP-TK at HPRT1 R Jx
ggtggaatacaactgcctgg

SEQ ID NO: 70 - DNA, 21 bp, Artificial Sequence
  forward primer for LP-PIGA at Sox2 L Jx
gactggggcttctcagagttc

SEQ ID NO: 71 - DNA, 18 bp, Artificial Sequence
  reverse primer for LP-PIGA at Sox2 L Jx
gggactgtgggcgatgtg

SEQ ID NO: 72 - DNA, 19 bp, Artificial Sequence
  forward primer for LP-PIGA at Sox2 R Jx
acccacagcttctcaacgg

SEQ ID NO: 73 - DNA, 20 bp, Artificial Sequence
  reverse primer for LP-PIGA at Sox2 R Jx
agtgactgcagcagacttgg

SEQ ID NO: 74 - DNA, 25 bp, Artificial Sequence
  forward primer for Ori
tttccataggctccgcccccctgac

SEQ ID NO: 75 - DNA, 20 bp, Artificial Sequence
  reverse primer for Ori
gttaccggataaggcgcagc

SEQ ID NO: 76 - DNA, 21 bp, Artificial Sequence
  forward primer for Sox2[Bl6]-5′
gactggggcttctcagagttc

SEQ ID NO: 77 - DNA, 24 bp, Artificial Sequence
  reverse primer for Sox2[Bl6]-5′
gatctctggtgtaccagtgtgtcc

SEQ ID NO: 78 - DNA, 21 bp, Artificial Sequence
  forward primer for PL1 at HPRT1
cctgatctgggtgactctagg

SEQ ID NO: 79 - DNA, 20 bp, Artificial Sequence
  reverse primer for PL1 at HPRT1
agaggttcagcagtgggaag

SEQ ID NO: 80 - DNA, 20 bp, Artificial Sequence
  forward primer for PL1 at Sox2 R Jx
agaatagcaggcatgctggg

SEQ ID NO: 81 - DNA, 20 bp, Artificial Sequence
  reverse primer for PL1 at Sox2 R Jx
agtgactgcagcagacttgg

SEQ ID NO: 82 - DNA, 20 bp, Artificial Sequence
  forward primer for MC L Jx
gaggcagggcaatcagaagt

SEQ ID NO: 83 - DNA, 20 bp, Artificial Sequence
  reverse primer for MC L Jx
agaatagcaggcatgctggg

SEQ ID NO: 84 - DNA, 17 bp, Artificial Sequence
  forward primer for Sox246kb at Sox2 R Jx
gggagggggccctgccg

SEQ ID NO: 85 - DNA, 20 bp, Artificial Sequence
  reverse primer for Sox246kb at Sox2 R Jx
agtgactgcagcagacttgg

SEQ ID NO: 86 - DNA, 21 bp, Artificial Sequence
  forward primer for Sox2143kb at Sox2 L Jx
gactggggcttctcagagttc

SEQ ID NO: 87 - DNA, 18 bp, Artificial Sequence
  reverse primer for Sox2143kb at Sox2 L Jx
gggactgtgggcgatgtg

SEQ ID NO: 88 - DNA, 21 bp, Artificial Sequence
  forward primer for Sox2143kb at Sox2 R Jx
agacttgttttcctcctgcct

SEQ ID NO: 89 - DNA, 17 bp, Artificial Sequence
  reverse primer for Sox2143kb at Sox2 R Jx
gccactgagaccgaggt

SEQ ID NO: 90 - DNA, 19 bp, Artificial Sequence
  forward primer for LP-PIGA at Sox2 R Jx
acccacagcttctcaacgg

SEQ ID NO: 91 - DNA, 20 bp, Artificial Sequence
  reverse primer for LP-PIGA at Sox2 R Jx
agtgactgcagcagacttgg

SEQ ID NO: 92 - DNA, 21 bp, Artificial Sequence
  forward primer for WT PIGA
ttactatctggcagggaaggc

SEQ ID NO: 93 - DNA, 20 bp, Artificial Sequence
  reverse primer for WT PIGA
ccatgcgtcacagctggtac

SEQ ID NO: 94 - DNA, 21 bp, Artificial Sequence
  forward primer for delta PIGA
ttactatctggcagggaaggc

SEQ ID NO: 95 - DNA, 23 bp, Artificial Sequence
  reverse primer for delta PIGA
tgtgatgggcataaaaggctact

SEQ ID NO: 96 - DNA, 21 bp, Artificial Sequence
  forward primer for WT HPRT1
ttcccagcaacaaagtaggag

SEQ ID NO: 97 - DNA, 20 bp, Artificial Sequence
  reverse primer for WT HPRT1
acaaggccaacagcagtctg

SEQ ID NO: 98 - DNA, 20 bp, Artificial Sequence
  forward primer for LP-PIGA at HPRT1 L Jx
tggtgcgatctcagctcagt

SEQ ID NO: 99 - DNA, 20 bp, Artificial Sequence
  reverse primer for LP-PIGA at HPRT1 L Jx
ttacgtctgctgcaggcgcg

SEQ ID NO: 100 - DNA, 19 bp, Artificial Sequence
  forward primer for LP-PIGA at HPRT1 R Jx
acccacagcttctcaacgg

SEQ ID NO: 101 - DNA, 20 bp, Artificial Sequence
  reverse primer for LP-PIGA at HPRT1 R Jx
acaaggccaacagcagtctg

SEQ ID NO: 102 - DNA, 20 bp, Artificial Sequence
  forward primer for LP Jx
atgagaacccggaaagaggg

SEQ ID NO: 103 - DNA, 20 bp, Artificial Sequence
  reverse primer for LP Jx
ccaacttctcggggactgtg

SEQ ID NO: 104 - DNA, 20 bp, Artificial Sequence
  forward primer for L Jx 1
atgagaacccggaaagaggg

SEQ ID NO: 105 - DNA, 20 bp, Artificial Sequence
  reverse primer for L Jx 1
agaatagcaggcatgctggg

SEQ ID NO: 106 - DNA, 20 bp, Artificial Sequence
  forward primer for L Jx 2
atgagaacccggaaagaggg

SEQ ID NO: 107 - DNA, 24 bp, Artificial Sequence
  reverse primer for L Jx 2
gatctctggtgtaccagtgtgtcc

SEQ ID NO: 108 - DNA, 17 bp, Artificial Sequence
  forward primer for R Jx
gggagggggccctgccg

SEQ ID NO: 109 - DNA, 20 bp, Artificial Sequence
  reverse primer for R Jx
gaggtctttggaaggcatgg

SEQ ID NO: 110 - DNA, 20 bp, Artificial Sequence
  forward primer for duplication
ccactcctgccacttcagag

SEQ ID NO: 111 - DNA, 20 bp, Artificial Sequence
  reverse primer for duplication
catccaaagggtgcaaaggc

SEQ ID NO: 112 - DNA, 20 bp, Artificial Sequence
  forward primer for deletion
ggaaaggcggcaactgtttt

SEQ ID NO: 113 - DNA, 20 bp, Artificial Sequence
  reverse primer for deletion
cgacgtttgaacgggtttcc

SEQ ID NO: 114 - DNA, 24 bp, Artificial Sequence
  forward primer for CreERT2 Fig. 6b)
cgattgatttacggcgctaaggat

SEQ ID NO: 115 - DNA, 21 bp, Artificial Sequence
  reverse primer for CreERT2 Fig. 6b)
gccatcttccagcaggcgcac

SEQ ID NO: 116 - DNA, 25 bp, Artificial Sequence
  forward primer for CreERT2 (Fig. 9b)
aatggtttcccgcagaacctgaaga

SEQ ID NO: 117 - DNA, 21 bp, Artificial Sequence
  reverse primer for CreERT2 (Fig. 9b)
agcaatccccagaaatgccag

SEQ ID NO: 118 - DNA, 20 bp, Artificial Sequence
  forward primer for Human HPRT1
gaccagtcaacaggggacat

SEQ ID NO: 119 - DNA, 20 bp, Artificial Sequence
  reverse primer for Human HPRT1
cctgaccaaggaaagcaaag

SEQ ID NO: 120 - DNA, 33 bp, Artificial Sequence
  forward primer for Human mini PIGA (hmPIGA)
gcctgattgaaagagggcataaggttataattg

SEQ ID NO: 121 - DNA, 26 bp, Artificial Sequence
  reverse primer for Human mini PIGA (hmPIGA)
gactggttgtacatgactttcagagg

SEQ ID NO: 122 - DNA, 20 bp, Artificial Sequence
  forward primer for Human total PIGA
tttgctgatgtcagctcggt

SEQ ID NO: 123 - DNA, 20 bp, Artificial Sequence
  reverse primer for Human total PIGA
ccaaaagacgcaccctgtca

SEQ ID NO: 124 - DNA, 20 bp, Artificial Sequence
  forward primer for Mouse Hprt1
aagcctaagatgagcgcaag

SEQ ID NO: 125 - DNA, 19 bp, Artificial Sequence
  reverse primer for Mouse Hprt1
ggacgcagcaactgacatt

SEQ ID NO: 126 - DNA, 14 bp, Artificial Sequence
  forward primer for Mouse Sox2[BL6]
aaccgatgcaccgc

SEQ ID NO: 127 - DNA, 20 bp, Artificial Sequence
  reverse primer for Mouse Sox2[BL6]
gagcattatcagatttttcc

SEQ ID NO: 128 - DNA, 14 bp, Artificial Sequence
  forward primer for Mouse Sox2[Cast]
agccgatgcaccga

SEQ ID NO: 129 - DNA, 21 bp, Artificial Sequence
  reverse primer for Mouse Sox2[Cast]
tgagcattatcagatttttct

SEQ ID NO: 130 - DNA, 20 bp, Artificial Sequence
  forward primer for BSD
tcgcgacgatacaagtcagg

SEQ ID NO: 131 - DNA, 20 bp, Artificial Sequence
  reverse primer for BSD
ggaccttgtgcagaactcgt

SEQ ID NO: 132 - DNA, 31 bp, Artificial Sequence
  HAL and surrounding genomic sequences, expected
attctcattttttagatgaagagactgaggt

SEQ ID NO: 133 - DNA, 31 bp, Artificial Sequence; identical to SEQ ID NO: 132
  HAL and surrounding genomic sequences, observed

SEQ ID NO: 134 - DNA, 31 bp, Artificial Sequence
  HAL and surrounding genomic sequences and LP- TK, expected
aagcaacgcgcgccaacgataacttcgtata

SEQ ID NO: 135 - DNA, 31 bp, Artificial Sequence; identical to SEQ ID NO: 134
  HAL and surrounding genomic sequences and LP- TK, observed

SEQ ID NO: 136 - DNA, 31 bp, Artificial Sequence
  HAR and surrounding genomic sequences and LP- TK, expected
ttatacgaagttattatgttgaggtagatgt

SEQ ID NO: 137 - DNA, 31 bp, Artificial Sequence; identical to SEQ ID NO: 136
  HAR and surrounding genomic sequences and LP- TK, observed

SEQ ID NO: 138 - DNA, 30 bp, Artificial Sequence
  HAR and surrounding genomic sequences, expected
agtgcaggtccagagaggtcacataatttg

SEQ ID NO: 139 - DNA, 30 bp, Artificial Sequence; identical to SEQ ID NO: 138
  HAR and surrounding genomic sequences, observed

(19) United States
(12) Patent Application Publication
(10) Pub. No.: US 2023/0392170 A1
(43) Pub. Date: Dec. 7, 2023

(54) BIG-IN: A VERSATILE PLATFORM FOR LOCUS-SCALE GENOME REWRITING AND VERIFICATION

(71) Applicant: New York University, New York, NY (US)

(72) Inventors: Ran BROSH, Forest Hills, NY (US); Jon LAURENT, New York, NY (US); Matthew MAURANO, New York, NY (US); Jef D. BOEKE, New York, NY (US)

(21) Appl. No.: 18/248,453

(22) PCT Filed: Oct. 14, 2021
(86) PCT No.: PCT/US2021/054988

§ 371 Date: Apr. 10, 2023

Related U.S. Application Data
(60) Provisional application No. 63/091,508, filed on Oct. 14, 2020.

(51) Int. Cl. C12N 15/90; C12N 15/10; C12N 5/0735; C12N 15/11; C12N 9/22; C12N 9/12

(52) U.S. Cl. CPC C12N 15/907; C12N 15/1082; C12N 5/0606; C12N 15/11; C12N 9/22; C12N 9/1211; C12Y 207/01021; C12N 2310/20; C12N 2800/80

(57) ABSTRACT

Provided are compositions and methods for using the compositions to modify eukaryotic chromosomes. The methods involve iteratively inserting DNA payloads into a chromosomal locus, or into multiple chromosomal loci. The methods utilize positive and negative selection approaches in combination with one or more recombinases to select cells that contain a payload, eliminate cells that do not contain a payload, and sequentially replace contiguous segments of the chromosome with subsequent payload insertions. Modified cells, and modified mammals containing modified cells, are included.

### CROSS-REFERENCE TO RELATED APPLICATIONS

This application claims priority to U.S. provisional application No. 63/091,508, filed Oct. 14, 2020, the entire disclosure of which is incorporated herein by reference.

### STATEMENT REGARDING FEDERALLY SPONSORED RESEARCH

This invention was made with government support under grant no. RM1-HG009491 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy was created on Oct. 13, 2021, is named “058636_00389_ Sequence_Listing_ST25.txt” and is 28,205 bytes in size.

### BACKGROUND

A global understanding of genomic regulatory architecture is critical to interpreting the effect of variants associated with common human traits and diseases¹. As the regulation of genes throughout development depends strongly on their native chromatin and genomic environments², short artificial constructs are inherently incapable of modeling the complexity of native loci, even when integrated genomically. Analysis of natural sequence variation in regulatory DNA provides one high-throughput approach for functional assessment in an endogenous cellular and genomic context, but detailed investigation of locus architecture is limited by the low frequency of informative variants and patterns of linkage disequilibrium^{3,4}.

Transgenic mammalian cell lines and animals generated using homologous recombination^{5,6} and the subsequent development of nuclease-mediated genome editing⁷ have enabled detailed functional analysis of the regulation of individual genes at their endogenous loci. These technologies have since facilitated screens of noncoding regulatory elements^{8,9} and locus-scale analyses^{10,11}. However, editing approaches offer limited control over the final sequence, a low maximum edit size, no inherent allele specificity at diploid loci, and the risk of off-target editing by designer nucleases¹².

Many limitations of genome editing do not apply to production of DNA using recombineering or yeast assembly approaches^{13,14}. Indeed, transgenesis of large constructs such as yeast and bacterial artificial chromosomes (YACs and BACs)¹⁵ has enabled position-independent, copy-number dependent expression, reproduction of organismal phenotypes such as the developmental switch from fetal to adult hemoglobin^{16,17}, and modeling of disease-associated variation¹⁸. Engineering of mammalian cells using recombinase-mediated cassette exchange (RMCE)¹⁹⁻²² or serine recombinase approaches' have enabled efficient single-copy targeting. RMCE schemes have been adapted for targeting large DNAs in mammalian cells^{24,25}. However, existing schemes are not readily portable to new loci or cell lines, in particular to stem cells which may not tolerate certain selection schemes. Furthermore, the gene traps employed to select for integrants remain as transcriptionally active genomic scars, which confound dissection of regulatory sequences unless removed through a subsequent engineering step. Finally, all these approaches suffer from the difficulty of verifying both on-target and off-target events. These technical limitations on editing endogenous loci have impeded the development of synthetic regulatory genomics as an approach to understanding the regulatory architecture of mammalian genomes.

Thus, there is an ongoing and unmet need for improved approaches to locus-scale genome modification. The disclosure is pertinent to these and other needs.

### BRIEF SUMMARY

The present disclosure relates generally to modifying chromosomes of eukaryotic cells, and in particular, mammalian cells. The method generally comprises iterative gene writing by sequential introduction of particular DNA segments into any genomic locus of interest. The compositions and methods include use of selection and counter selection to provide for insertion of large DNA segments, e.g., up to 5 kilobases (kb), or more. The DNA segments include a payload segment that can code for and facilitate expression of any RNA, including mRNA, and the concomitant expression of the protein encoded by the mRNA. The compositions and methods are suitable for modifying any mammalian cells. The modifications can be homozygous, heterozygous, or hemizygous. The cells modified using the described compositions and methods may be haploid, diploid, or tetraploid. The compositions and methods can thereby result in modified cells. The modified cells may comprise any type of stem cells, specific examples of which are discussed in the detailed description. The modified stem cells can be used to produce modified embryos, and modified mammals that develop from the modified embryos.

In one aspect, the disclosure provides a method for insertion of a DNA payload into a chromosomal locus in mammalian cells. The method generally comprises introducing into a selected locus a first double stranded DNA template (referred to herein as a landing pad “LP”) that comprises 5′ and 3′ homology arms (HAs). The LP comprises one or more selection markers. The LP comprises a pair of recombinase recognition sites configured to excise a segment of the LP that comprises at least one negative selection marker. The method comprises selecting cells that comprise the LP using the positive selection marker to obtain an isolated population of the mammalian cells that comprise the LP. Once selected cells that comprise the LP are selected, the method further comprises introducing into the selected cells a second dsDNA comprising a payload sequence and a positive selection marker used to select cells that comprise the payload. The positive selection marker is i) within the payload sequence in the second dsDNA and is inserted into the locus, or ii) is present on a location on the second dsDNA that is not inserted into the locus. A recombinase that is introduced into or already present in the cells recognizes the recombinase recognition sites and removes at least the segment of the LP that comprises the negative selection marker in at least some of the mammalian cells, such that at least the segment of the LP comprising the negative selection marker is replaced by the payload by homologous recombination of the payload into the locus in at least some of the mammalian cells. The method further comprises exposing the mammalian cells to an agent that acts on the negative selection marker such that only mammalian cells that contain the LP and the negative selection marker but not the payload are killed. Subsequently, the method comprises separating mammalian cells that comprise the payload but do not contain the LP to thereby obtain isolated viable mammalian cells that comprise the payload.

The LP may be introduced into the mammalian cells using any of a variety of techniques, which include by are not necessarily limited to using a nuclease system selected from an RNA-guided clustered regularly interspaced short palindromic repeats (CRISPR) enzyme, a Transcription activator-like effector nuclease (TALEN), a zinc finger nuclease, or a MAD-series nuclease.

In non-limiting embodiments, the mammalian cells into which the LP is introduced comprise an endogenous mutated gene that encodes Phosphatidylinositol Glycan Anchor Biosynthesis Class A (PIGA) enzyme such that the function of the PIGA enzyme is reduced or eliminated relative to a non-mutated gene that encodes the PIGA enzyme. In this configuration, the LP comprises a sequence encoding a functional PIGA enzyme as a negative selection marker, wherein an agent that acts on the negative selection marker is used and comprises Proaerolysin. In embodiments, the LP comprises a sequence encoding a herpes simplex virus type 1—thymidine kinase (HSV1-TK). In this configuration, an agent that acts on the negative selection marker is ganciclovir.

In various embodiments, the payload is only inserted into the locus on one homologous chromosome to thereby provide a heterozygous chromosome pair in which only one chromosome in the pair comprises the payload. In an embodiment, a positive selection marker is within the payload sequence in a second dsDNA and is inserted into the locus with the payload. In embodiments, the positive selection marker is present on a location on a second dsDNA that is not inserted into the locus, and the payload is inserted into the locus without the positive selection marker.

The disclosure also includes mammalian cells that are made using the described compositions and methods. The disclosure includes modified stem cells, and embryos that comprise the modified stem cells. Non-human transgenic mammals made by the described compositions and methods are also included.

### BRIEF DESCRIPTION OF THE FIGURES

FIG. 1. Landing pad integration in human and mouse ESCs. a, Replacement of the 42 kb HPRT1 locus in H1 hESCs with a landing pad (LP-TK) utilizing CRISPR/Cas9 and 1 kb HAs (gray). Cells are selected for LP-TK presence with puromycin and HPRT1 inactivation with 6-TG. b, PCR genotyping of H1 clones for novel left (L) and right (R) junctions (Jx) using primers illustrated in a. Par, parental H1. c, Sequencing verification pipeline using whole genome sequencing (WGS) or targeted libraries. Capture-Seq enriches for regions of interest using biotinylated bait prepared using nick translation from relevant DNA constructs. d, WGS of parental H1 hESCs and LP-TK clone 581 mapped to hg38 shows the 42 kb deletion of the HPRT1 locus. e, Mapping to LP-TK (left) and LP-TK backbone (right) confirms specific gain of LP-TK; regions cross-mapping with human genome are shaded light gray (pEF1α, EEF1A1 promoter; ERT2, ESR1 ligand binding domain⁵⁶; pA, EIF1 pA signal). f, Mapping to pCas9 confirms plasmid loss; regions shaded light gray cross-map with human (pU6, U6 promoter) and LP-TK (PuroR, puromycin resistance gene). g, Replacement of a 143 kb region of the Sox2 locus on the BL6 allele of chromosome 3 (black) in BL6xCAST mESCs with of LP-PIGA utilizing CRISPR/Cas9, facilitated by 0.15 kb HAs (gray). h, Top and bottom left, Screening of BL6xCAST LP-PIGA clones using PCR genotyping primers targeting novel junctions illustrated in i. Bottom right, secondary screening of 16 clones positive for both junctions using primers for plasmid origin of replication (Ori) and the BL6 Sox2 allele (Sox2[BL6]). 7 clones marked with asterisks had the desired genotype; Clone A1 was selected for further analysis. Par, parental BL6xCAST mESCs. L, ladder. i-k, Capture-Seq analysis of parental BL6xCAST mESCs, LP-PIGA clone A1, and an example failed clone from an independent LP-PIGA delivery. Reads were mapped to the references indicated above. Cross-mapping sequences are shaded light gray.

FIG. 2. Development of an efficient counterselection strategy. a, Parental (TK−) and LP-TK (TK+) H1 hESCs were co-cultured at the indicated ratios, treated with 1 μM, GCV for 4 days, and assayed for the number of live cells using PrestoBlue. Cell counts are shown relative to unmixed parental cells. Bars show mean+S.D. (n=2). b, GCV enters TK+ cells and is metabolized into the toxic membrane-impermeable compound GCV-TP, which diffuses into neighboring cells and induces bystander cell death in TK-cells. c, Big-IN counterselection strategy using PIGA/Proaerolysin. d, Parental and ΔPIGA H1 hESCs co-cultured at the indicated ratios for three days were treated with 1 nM proaerolysin for 1 day and stained with Crystal Violet 3 days later. e, Parental and ΔPiga BL6xCAST mESCs were co-cultured at the indicated ratios for one day, treated with 1 nM proaerolysin for 1 day, stained with Crystal Violet 5 days later, and colonies were counted. Bars represent mean+S.D. (n=2).

FIG. 3. Efficient delivery to H1 hESCs. a, LP-TK at HPRT1 undergoes recombinase-mediated cassette exchange with PL1 following transfection and Cre induction. Payload integration can be selected for with blasticidin and GCV. BSD, Blasticidin S deaminase. b, Genotyping of untransfected LP-TK hESCs (clone 581), PL1-transfected pool, and clones using PCR primers flanking payload lox sites (illustrated in a). All clones produced the expected 3 kb product (a 5.7 kb product for LP-TK cells was not detected at this extension time). c, Capture-Seq analysis of chosen H1 PL1 clones mapped to PL1 (left) and its backbone (right). d, Capture-Seq reads mapped to LP-TK, validating LP loss in PL1 clones. Cross-mapping sequences are shaded light gray.

FIG. 4. Efficient delivery to mESCs. a, Delivery of three payloads to BL6xCAST LP-PIGA mESCs. b, PCR genotyping of PL1 (top) and Sox2^{46kb}-MC (bottom) mESC clones for the novel junctions illustrated in a. L, ladder; E, empty well. c-d, Capture-Seq analysis of chosen PL1 and Sox2^{46kb}-MC mESC clones, with Parental and LP-PIGA mESCs as controls. c. Sequencing coverage mapped to PL1. pEF1α (shaded light gray) is present in both LP-PIGA and PL1. d, Gain of coverage in Sox2^{46kb}-MC mESCs at the 46 kb payload region. Black ticks under each coverage track indicate detection of BL6 alleles at known SNVs. Internal payload duplication marked in Clone C9 (see FIG. 12). e, PCR genotyping of Sox2^{143kb} clones for the novel junctions using BL6-specific primers, and for loss of LP-PIGA, as illustrated in a. f, Sox2^{143kb} mESCs show restored coverage of the full 143 kb genomic region corresponding to the payload. Black ticks under each coverage track indicate detection of BL6 alleles at known SNVs. Coverage at right shows no retention of payload backbone; cross-mapping sequences are shaded light gray. g, qRT-PCR expression analysis of Sox2^{143kb} clone G11 and LP-PIGA mESCs for mRNAs from BL6 and CAST Sox2 alleles, payload-derived Blasticidin-S deaminase (BSD), and LP-harbored hmPIGA. Bars represent mean+S.D. for technical replicates (n=3).

FIG. 5. bamintersect, a tool for integration site analysis. a, Schematic of the bamintersect analysis pipeline. b-h, bamintersect results between genomic and custom references indicated at top of each panel. Bars represent the number of reads supporting each junction, normalized to 10 million sequenced reads. Junctions were annotated as expected left, expected right, or off-target. For PL1 integration at both HPRT1 and Sox2 (b and c), the expected left junction is not shown due to its near identity with LP being replaced. For integrations at Sox2 (b, e and f), the expected left junction is adjacent to a low mappability region composed of simple repeats and an Alu sequence, consistently yielding fewer reads relative to the right junction. Allelic analysis in f categorizes reads at expected left and right junctions using known BL6xCAST SNVs; uninformative reads do not overlap known variants.

FIG. 6. Targeted locus-scale genome rewriting using Big-IN. An allele of interest is replaced by a LP using CRISPR/Cas9-mediated HDR. A pair of gRNAs target the termini of the replaced allele and the LP, and short homology arms mediate precise LP integration. Puromycin selects for LP-harboring cells. Next, Cre-mediated recombination of two pairs of heterotypic loxM and loxP sites results in LP/Payload cassette exchange and resistance to GCV for HSV1-ΔTK or Proaerolysin for hmPIGA. Positioning the blasticidin cassette (BSD) within the payload permits election for high-efficiency integration; positioning BSD on the payload backbone permits transient selection for scarless delivery. Additionally, backbone HSV-ΔTK (left) can be counterselected with GCV to limit off-target integration. Each engineering step is comprehensively verified by PCR genotyping, WGS or Capture-Seq, and functional assays.

FIG. 7. Landing Pad Integration in human and mouse ESCs. a, Sanger sequencing of junction PCR products for H1 LP-TK clone 581 (FIG. 1, panel b) showing junctions between HAs and surrounding genomic sequences or LP-TK. Top, expected sequence; bottom, observed sequence and chromatograms. Segment i. Top, HAL and surrounding genomic sequences, expected (SEQ ID NO:132); Bottom, HAL and surrounding genomic sequences, observed (SEQ ID NO:133) Segment ii. Top, HAL and surrounding genomic sequences and LP-TK, expected (SEQ ID NO:134); Bottom, HAL and surrounding genomic sequences and LP-TK, observed (SEQ ID NO:135) Segment iii. Top, HAR and surrounding genomic sequences and LP-TK, expected (SEQ ID NO:136); Bottom, HAR and surrounding genomic sequences and LP-TK, observed (SEQ ID NO:137) Segment iv. Top, HAR and surrounding genomic sequences, expected (SEQ ID NO:138); Bottom, HAR and surrounding genomic sequences, observed (SEQ ID NO:139) b, qRT-PCR mRNA measurement of H1 LP-TK clone 581. Data is normalized to parental H1 hESCs and to the expression of GAPDH. Bars represent mean+S.D. of technical replicates (n=3). c, H1 LP-TK clone 581 and non-engineered parental H1 hESCs were treated with the indicated concentrations of GCV for 3 days, followed by quantification of live cells using PrestoBlue. Data were normalized to the value of untreated cells. Bars represent mean±S.D. of technical replicates (n=2). d, Lentiviral reporter wherein Cre activity results in DsRed excision (floxing) and GFP expression. LP-TK clone 581 hESCs and Parental H1 hESCs (Par) transduced with reporter were treated with varying concentrations of 4-Hydroxytamoxifen (Tam) and assayed by flow cytometry 2 days later. e, Effect of HA length on LP-TK integration at HPRT1. H1 hESCs were transfected with LP-TK plasmids differing only by their HA lengths, and with pCas9 HPRT1-g1 and HPRT1-g2. Cells were selected with puromycin 2 days post-transfection for 4 days and then with puromycin and 6-TG for an additional 3 days. Relative cell number was measured using PrestoBlue following puromycin selection (puro), which selects for any LP integration event, or combinatorial puromycin and 6-TG selection, which selects for on-target integration. Bars represent mean+S.D. of technical replicates (n=2). f, The effect of in vivo pLP linearization on backbone integration. H1 hESCs were transfected with a pLP-TK, containing or lacking gRNA binding sites corresponding to the cotransfected pCas9 HPRT1-g1 and HPRT1-g2. Cells were selected with puromycin 1 day post transfection for 5 days and puromycin+6-TG for an additional 5 days and were then subjected to Capture-Seq analysis. g, qRT-PCR mRNA analysis for BL6xCAST LP-PIGA clone A1 cells. Values are normalized to parental cells and to the expression of either Gapdh (for Cre^{ERT2} and hmPIGA) or Hprt1 (for Sox2). Bars represent mean+S.D. of technical replicates (n=3).

FIG. 8. Development of an efficient counterselection strategy. a, Lack of paracrine activity in GCV/TK bystander effect. Parental H1 cells were grown for 7 days with regular StemFlex medium with or without 0.1 μM GCV or with a 1:1 mixture of regular medium and conditioned medium harvested from LP-TK cells grown with or without 0.1 μM GCV. While GCV kills LP-TK cells (FIG. 7, panel c), it had no effect on parental cells, even when preincubated with LP-TK cells, suggesting lack of a paracrine effect and supporting a gap junction intercellular communication mechanism. Bars represent mean+S.D. for technical replicates (n=3). b, PCR genotyping for parental (Par), ΔPIGA and LP-PIGA H1 hESCs. Primers target a region of the PIGA gene (WT PIGA), the novel junction formed following PIGA deletion (ΔPIGA), a region of HPRT1 gene (WT HPRT1) and the two novel junctions formed between LP-PIGA and the surrounding genome (LP L and R Jx). c, Proaerolysin-induced cell death. The indicated H1 hESCs were treated with 0.5 nM proaerolysin for 1 day and stained with Crystal Violet. Proaerolysin resistance is conferred by PIGA inactivation and restored by hmPIGA expression. d, Rapid proaerolysin-induced cell death. e, Proaerolysin kill-curve for H1 hESCs and BL6xCAST mESCs. Cells were treated with proaerolysin for 1 day and assayed 3 days (H1) or 1 day (BL6xCAST) post treatment. Experiment was conducted in replicates (H1, n=4; BL6xCAST, n=2) and data are represented as mean±S.D. f, CD59/HLA flow cytometry of H1 hESCs. Note the complete loss of CD59, a GPI-anchored protein, in ΔPIGA cells, and the retention of HLA, a non-GPI-anchored membranal protein. g, Flow cytometry of H1 hESCs showing the reconstitution of CD59 expression in LP-PIGA cells.

FIG. 9. Transcriptional silencing of landing pad expression in the absence of positive selection. a, CD59 flow analysis of H1 hESCs grown with or without (w/o) puromycin for 12 days. The percent of CD59-negative (PIGA-inactivated) cells is indicated. b, qRT-PCR analysis of mRNA expression of H1 hESCs grown with or without puromycin for 12 days. Total PIGA was measured using primers that target both the endogenous PIGA gene and the LP-expressed PIGA minigene (hmPIGA). Bars represent mean+S.D. of technical replicates (n=4). c, Acquisition of proaerolysin resistance by BL6xCAST LP-PIGA mESCs. 1×10⁶ cells, grown in the presence or absence of puromycin for the indicated number of days, were plated in one well of a 6-well plate and treated with 2 nM proaerolysin the next day. Cells were maintained in proaerolysin-containing medium until surviving cells formed visible colonies, which were then stained with Crystal Violet and counted.

FIG. 10. Efficient delivery to human and mouse ESCs. a, Brightfield and GFP microscopy images of chosen H1 PL1 clones. b, Capture-Seq analysis of a representative failed BL6xCAST Sox2^{46kb}-MC clone. Sequence coverage mapped to mm10 reveals gain of multiple Sox^{246kb}-MC payload copies (coverage corresponding to the payload region exceeds surrounding regions). Additionally, mapping to the payload backbone reveals its presence at a 1:1 stoichiometric ratio with the payload, while mapping to LP-PIGA shows its retention. Cross-mapping sequences are shaded gray. c, Sequencing coverage depth at the engineered Sox2 locus relative to flanking genomic regions in BL6xCAST PL1 and Sox2^{46kb}-MC clones; coverage is calculated for the region corresponding to the Sox2^{46kb} payload and for the remaining distal region (see b). An asterisk denotes increased depth for Sox2^{46kb}-MC clone C9 due to an internal payload partial duplication (see FIG. 4). d, Sequencing coverage depth normalized to total sequencing depth at the payload marker cassette and the payload vector backbone in BL6xCAST PL1 and Sox2^{46kb} clones. e, qPCR analysis for human PIGA DNA level in PL1 and Sox2^{46kb} BL6xCAST mESC colonies. Values are normalized to the level detected in LP-PIGA mESCs. Parental BL6xCAST mESCs (Par) and TE are included as negative controls. Bars represent median values. f, qRT-PCR analysis of mRNA expression of chosen BL6xCAST PL1 and pSox2^{46kb} clones and LP-PIGA mESCs. Measured mRNAs include the BL6 and CAST alleles of Sox2, detected using allele-specific primers and the payload-derived Blasticidin-S deaminase (BSD). Mean values were normalized to the expression of Gapdh and to parental cells. Bars represent mean+S.D. of technical replicates (n=3). g, Brightfield and GFP microscopy images of chosen BL6xCAST PL1 and Sox2^{46kb} mESC colonies.

FIG. 11. Payload delivery to the mouse Igf21H19 locus. a, BL6xCAST mESCs harboring LP-PIGA2 at the BL6 allele of Igf2/H19 were transfected with pSox2^{46kb}-MC-BBTK or pSox2^{46kb} and with pCAG-iCre (pCre). Transfected cells were selected with blasticidin, proaerolysin and GCV (for pSox2^{46kb}-MC-BBTK only). b, PCR verification of clones using junction (Jx) primers illustrated in a. LP, BL6xCAST LP-PIGA2 (at Igf2/H19) mESCs. c, Capture-Seq coverage of Sox2^{46kb} payload and surrounding region in mm10. Asterisks denote internal payload deletions detected in two clones. d, Sequencing coverage depth at Sox2 relative to flanking genomic regions demonstrates a 1.5-fold increase in coverage depth upon delivery of Sox2^{46kb}. Asterisks denote clones with internal payload deletions (see FIG. 12). e, Sequencing coverage depth normalized to total sequencing depth shown at LP-PIGA2, the BSD-T2A-GFP payload marker cassette, and the payload backbone.

FIG. 12. Detection of rearrangements in delivered DNA through Capture-Seq. a, Sequencing coverage for Parental, LP-PIGA and two Sox2^{46kb}-MC BL6xCAST clones showing increased depth over a 17 kb region in BL6xCAST Sox2^{46kb}-MC clone C9 (delivery to Sox2 locus, see FIG. 4) and reduced depth over a 7 kb region in BL6xCAST Sox2^{46kb}-MC clone A4 (delivery to Igf2/H19 locus, see FIG. 11). Red dashed lines denote mean coverage depth in flanking regions. b, Enlargement of payload region showing read pairs supporting rearrangements. Blue and red arrows represent PCR primers used to confirm rearrangements. c, PCR confirmation of rearrangement.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the effective filing date of this application or patent.

The disclosure in certain aspects provides for sequential modification of eukaryotic cells that results in substitution of an introduced landing pad (LP) with a DNA payload.

Generally, the method comprises modifying a chromosome by inserting the LP in a selected locus, and replacing all or a segment of the LP with the DNA payload. The LP can comprise negative and positive selection markers so that cells that initially comprise the LP can be selected, and cells that do not comprise the LP (and which may also comprise the payload) can be eliminated. Cells that contain the LP are modified by a one-step recombinase mediated insertion of the DNA payload, which may be large (e.g., greater than 100 kb in length). After introduction into the cells, the payload may provide a transient (e.g., in a plasmid backbone) or persistent (e.g., integrated) positive selection marker that allows selection of cells that contain the payload but do not contain the LP. The disclosure includes insertion of multiple payloads in the same locus, and insertion of the same or different payloads in different loci, including multiple copies thereof if desired. Thus, iterative cell editing is included. The disclosure includes scarless insertion of the payload, with the exception of retained recombinase recognition sequences, as further described below.

The methods of this disclosure are performed using DNA constructs and involve the participation of certain proteins. In embodiments, the protein may be produced within the cell via expression of any suitable expression system that encodes the protein. In embodiments, any protein required to participate in the described process may be modified such that it includes a nuclear localization signal. In embodiments, a protein may be administered directly to the cells. For proteins that require an RNA component to function, such as certain Cas proteins as described below, the protein(s) and the RNA component may be administered to the cells as ribonucleoproteins (RNPs).

### The LP

The disclosure in certain aspects provides for initial insertion of an LP into any desired chromosomal locus. In embodiments, the LP comprises first and second homology arms (each an “HA” and together “HAs”) which are configured to be introduced into any desired chromosomal locus using any suitable nuclease.

The sequence of the 5′ and 3′ homology arms are not particularly limited, provided they have a length that is adequate for homologous recombination to occur when nuclease-mediated cleavage of the selected locus occurs. In embodiments, the 5′ and 3′ homology arms have a length of from 100 bp-10 Kbp, inclusive, and including all integers and ranges of integers there between. In embodiments, the entire LP is 3.5 to inclusive, and including all integers and ranges of integers there between.

The LP includes recombinase recognition sequences that are configured so that a segment of the LP between the HAs can be recognized and excised by one or more recombinases in order to subsequently replace LP with the payload by operation of the recombinase.

The type of recombinase and recombinase recognition signals are not particularly limited, other than a preference for maintenance of the recombination recognition sites after a recombination event to enable iterative removal and insertion of different payloads in the same locus. Thus the disclosure includes using any site-dependent recombinase that recognizes heterotypic recombination sites.

In embodiments, the recombinase comprises Cre recombinase, and is used with lox sites, such as loxP and LoxM sites; or a Flp Recombinase which functions in the Flp/FRT system; or a Dre recombinase which functions in the Dre-rox system; or a Vika recombinase which functions in the Vika/vox system, or a BxB1 recombinase that functions with attP/attB sites. In embodiments, the recombinase can be provided to the cells in the form of a protein. In embodiments, the recombinase is encoded by an extrachromosomal element, such as a plasmid, or any other suitable vector, including but not limited to viral delivery vectors. The presence of the extrachromosomal element may be transient. In embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as a lentiviral vector. In embodiments, a recombinant adeno-associated virus (rAAV) vector may be used. rAAV vectors are commercially available, such as from TAKARA BIO® and other commercial vendors, and may be adapted for use with the described compositions and methods, given the benefit of the present disclosure. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure. In one embodiment, the recombinase is encoded by and expressed from the LP. Expression of the recombinase may be inducible. In embodiments, expression of the recombinase may be controlled by a repressor. In embodiments, expression of the recombinase may be from an inducible promoter that is operably linked to the sequence encoding the recombinase. The DNA sequences of a wide variety of inducible promoters for use in eukaryotic cells are known in the art, as are the agents that are capable of inducing expression from the promoters. In embodiments, engineered regulated promoters such as the Tet promoter TRE which is regulated by tetracycline, anhydrotetracycline or doxycline, or the lad-regulated promoter ADHi, which is regulated by IPTG (isopropyl-thio-galactoside) may also be used. In embodiments, the activity or localization of the recombinase can be regulated. These embodiments include but are not limited to the use of tamoxifen-based relocalization of a recombinase to the nucleus or ligand-induced dimerization of the enzyme. In embodiments, expression of the recombinase may be controlled by, for example, by a degron. In non-limiting embodiments, the degron is a component of a degron system, including ubiquitin-dependent and ubiquitin-independent degron systems.

Virtually any chromosomal locus can be a site for insertion of an LP. In embodiments, the LP is introduced into a selected locus using any designer nuclease. In embodiments, the nuclease is a RNA-guided CRISPR-associated (Cas) nuclease. A variety of suitable CRISPR nucleases (e.g., Cas nucleases) are known in the art, as are methods for designing and selecting appropriate guide RNA constructs so that HAs can be precisely integrated at a predetermined location using a Cas nuclease. Thus, in embodiments, an RNA-guided Cas nuclease may be used. In an embodiment, two guide RNAs may be included so that the locus is modified in two positions, one for each HA.

In embodiments, the Cas is selected from a Class 1 or Class 2 Cas enzyme. In embodiments, a Type II or a Type V CRISPR Cas is used. In specific and non-limiting embodiments, the Cas comprises a Cas9, such as Streptococcus pyogenes (SpCas9). Derivatives of Cas9 are known in the art and may also be used to introduce the LP into a locus. Such derivatives may be, for example, smaller enzymes than Cas9, and/or have different proto adjacent motif (PAM) requirements. In non-limiting embodiments, the Cas enzyme may be Cas12a, also known as Cpf1, or SpCas9-HF1, or HypaCas9. The first and second HAs can include sequences that are recognized and cleaved by the same Cas-mediated cleavage system that recognizes and cleaves the chromosomes, as described and illustrated further herein. This configuration is particularly useful when, for example, the LP is provided on a plasmid, whereby excision of the plasmid-based LP facilitates the liberation of the HAs to aid in homologous recombination into the chromosomes. Thus, this approach also linearizes the plasmid. Cas cleavage sites may be positioned at or near the end of the HA arms.

The LP may also be inserted into a selected locus using non-Cas based nuclease approaches. Suitable examples include but are not necessarily limited to zinc-finger nucleases and MADzymes. Non-limiting examples of MADzymes known in the art include MAD2 and MAD7 and are included in the Cas12a category of nucleases.

In embodiments, the LP comprises a nucleotide sequence that is also cleaved by the nuclease, which for example, may induce linearization of a plasmid that contains the LP.

In embodiments, the LP comprises selectable markers. In embodiments, the LP includes a negative selection marker (also referred to as a counterselection marker). In embodiments, the negative selection marker is operatively linked to a positive selection marker. Examples of suitable selection markers are known and can be adapted for use with the described compositions and methods by those skilled in the art when given the benefit of the present disclosure. Suitable examples of positive selection markers to obtain cells that initially include the LP, and in which the LP will be replaced by the payload as further described herein, include but are not limited to puromycin N-acetyltransferase (pac), Blasticidin S deaminase (bsd), Neomycin (G418) resistance gene (neo), Hygromycin resistance gene (hygB), and Zeocin resistance gene (Sh bla).

Non-limiting examples of negative selection markers include use of the HSV1-TK gene that renders cells sensitive to ganciclovir (GCV) by converting it to the toxic metabolite GCV-triphosphate (GCV-TP). HSV1-TK can also be used as a positive selection marker using HAT medium.

In another embodiment, the cells into which the LP are introduced may have a mutated X-linked PIGA (phosphatidylinositol glycan class A) gene. A mutation in the PIGA gene may be made by adapting strategies described further herein, including but not limited to CRISPR-mediated mutations that are produced using a suitable guide RNA(s). The protein encoded by the PIGA gene renders cells sensitive to the bacterial prototoxin proaerolysin. Thus, cells into which an LP is introduced may include a functional PIGA gene that encodes a protein that renders the cells sensitive to proaerolysin, which facilitates elimination of cells that include the LP, e.g., cells in which the LP is present but wherein the desirable cells are those in which the LP is replaced by the payload, as further described below. Thus, positive and negative selection facilitates selecting cells that contain the LP, and eliminating cells that contain the LP after the payload has been recombined into the cells.

### The Payload

In embodiments, the payload may comprise or consist of 1 bp-1,000 kb, inclusive, and including all numbers and ranges of numbers there between, and in certain instances may be longer than 1000 kb.

Without intending to be constrained by any particular theory, it is considered that, other than a requirement for certain sequences to function with the recombinase as described herein, the presently provided systems are ambivalent with respect to the DNA sequence of the DNA insertion template. Accordingly, in embodiments, the DNA insertion template may be devoid of any sequence that can be transcribed, and as such may be transcriptionally inert. Such sequences may be used, for example, to alter a regulatory sequence in a genome, e.g., a promoter, enhancer, miRNA binding site, or transcription factor binding site, to result in knockout of an endogenous gene, or to provide an interval in the chromosome between two loci, and may be used for a variety of purposes, which include but are not limited to treatment of a genetic disease, enhancement of a desired phenotype, study of gene effects, chromatin modeling, enhancer analysis, DNA binding protein analysis, methylation studies, and the like.

In embodiments, payload comprises a sequence that may be transcribed by any RNA polymerase, e.g., a eukaryotic RNA polymerase, e.g., RNA polymerase I, RNA polymerase II, or RNA polymerase III. In embodiments, the RNA that is transcribed may or may not encode a protein, or may comprise a segment that encodes a protein and a non-coding sequence that is functional, such as a functional mRNA.

In embodiments, the payload includes one or more promoters. The promoter may be constitutive or inducible. The promoter may be operably linked to a sequence that encodes any protein or peptide, or a functional RNA.

In embodiments, the payload comprises one or more splice junctions.

In embodiments, the payload comprises an intact gene, or a gene fragment. The payload may include one or more genes or gene fragments. The gene or gene fragments may contain exons an introns. In embodiments, the payload comprises a cluster of genes. In embodiments, the payload comprises any of the foregoing features, which may be operably linked to a promoter that is included within the payload, or the DNA insertion template is linked to an endogenous cell promoter once integrated. In embodiments, the payload comprises at least one open reading frame. In embodiments, the payload encodes a protein.

In embodiments, the protein encoded by payload encodes a binding partner, such as an antibody or antigen binding fragment of an antibody. In embodiments, one or more binding partners encoded by the payload may be all or a component of a Bi-specific T-cell engager (BiTE), a bispecific killer cell engager (BiKE), or a chimeric antigen receptor (CAR), such as for producing chimeric antigen receptor T cells (e.g. CAR T cells). In embodiments, the payload encodes a T cell receptor, and thus may encode both an alpha and beta chain T cell receptor.

In embodiments, the payload comprises a sequence that is intended to disrupt or replace a gene or a segment of a gene. Thus, the disclosure includes producing both knock in and knock out gene modifications in cells, and transgenic non-human animals that contain such cells.

In embodiments, the payload is used to modify one or more chromosomal loci that are involved in, for example, any genetic disease. The payload may differ from an endogenous gene by as little as a single nucleotide, or may include or lack a particular exon, a splice junction, etc. The payload may also be a completely new sequence, relative to the genome of the cell prior to using the described approached to modify one or more loci. In embodiments, a detectable marker is encoded by the payload.

In embodiments, the payload comprises, as noted above, a positive selection marker, which may be present only during selection of the cells, or may be encoded by the payload, the former configuration allowing for scar-less insertion of the payload, except for the remainder of the recombinase recognition sequences.

### Modified Cells

In general, the described approaches are used to modify eukaryotic cells. The modified locus may be in the nucleus.

In embodiments, the eukaryotic cells comprise animal cells, which may comprise mammalian or avian cells, or insect cells. In embodiments, the mammalian cells are human or non-human mammalian cells. In embodiments, the cells are from avian animals, a canine, a feline, an equine animal, a murine mammal, e.g. a mouse or rat, a ruminant, or a psuedoruminant.

In embodiments, the cells that are modified by the approaches of this disclosure are totipotent, pluripotent, multipotent, or oligopotent stem cells when the modification is made. The stem cells may exhibit the described potency naturally, or the stem cells may be induced stem cells. In embodiments, the cells are hematopoietic stem cells. In embodiments, the cells are embryonic stem cells, or adult stem cells. In embodiments, the cells are epidermal stem cells or epithelial stem cells or neural stem cells. In embodiments, the cells are cancer cells, or cancer stem cells. In embodiments, the cells are spermatogonial stem cells. In embodiments, the cells are differentiated cells when the modification is made. In embodiments, the cells are leukocytes. In embodiments, the leukocytes are of a myeloid or lymphoid lineage. In embodiments, cells modified according to the compositions and methods of this disclosure are haploid, diploid, or tetraploid.

In embodiments, the disclosure includes obtaining cells from an individual, modifying the cells ex vivo using a system as described herein, and reintroducing the cells or their progeny into the individual or an immunologically matched individual for prophylaxis and/or therapy of a condition, disease or disorder. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, eukaryotic cells made according to this disclosure can be used to create transgenic, non-human organisms. In embodiments, a non-human mammal may be modified to include one or more human genes. In embodiments, the disclosure comprises modifying a gene in a mammalian embryo, such as a disease causing or disease associated gene, and implanting the embryo into a mammalian female.

In embodiments, one or more modified cells according to this disclosure may be used to perform a gene-drive in a population of animals, including but not necessarily limited to insects.

In embodiments, the one or more cells into which a described system is introduced comprises a plant cell, including but not limited cells from any variety of cannabis, tobacco, maize, rice, ornamental and vegetable plants.

In embodiments, the disclosure comprises providing a treatment to an individual in need thereof by introducing a therapeutically effective amount of modified eukaryotic cells as described herein to the individual, such that the payload produces a polynucleotide, peptide, protein, a drug, a prodrug, an immunological agent, an enzyme, or any other agent that may have a beneficial effect. A corrected or new gene may also be considered a therapeutic agent.

In embodiments, the modified eukaryotic cells can be provided in a pharmaceutical formulation, and such formulations are included in the disclosure. A pharmaceutical formulation can be prepared by mixing the modified eukaryotic cells with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference.

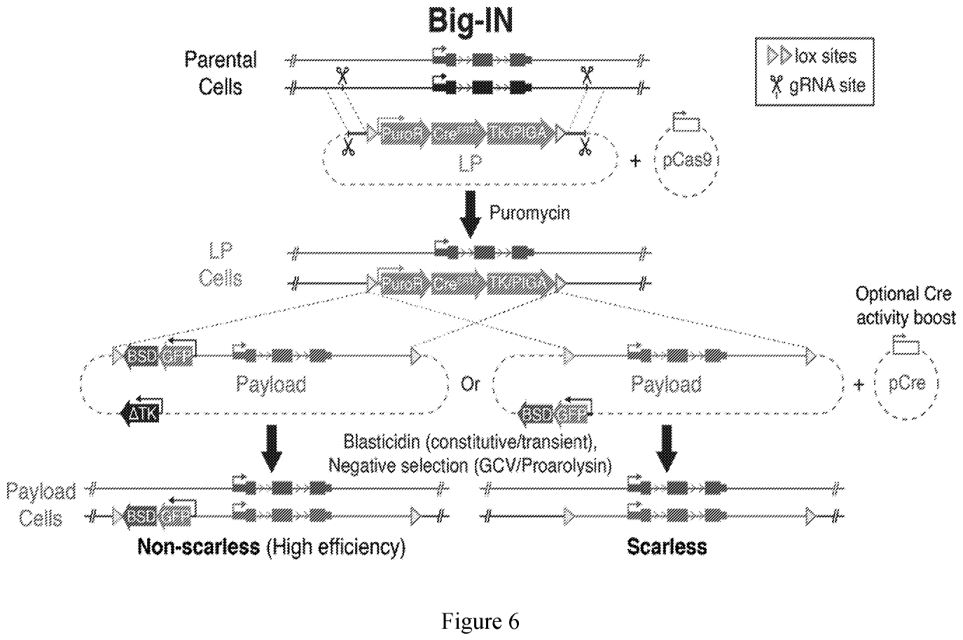

The Examples below are intended to illustrate but not limit the disclosure. The Examples show, among other aspects of the disclosure, that the present disclosure a platform for scalable targeted integration into mammalian genomes, and demonstrated its flexibility, efficiency, and precision at three loci in mouse and human embryonic stem cells. Big-IN first targets a landing pad to a locus of interest using CRISPR-Cas9-mediated HDR, which permits single-step payload integration through Cre-mediated RMCE (FIG. 6). The single-step payload integration minimizes confounding technical factors by permitting repeated deliveries to the same allele, and is thus ideal for high-throughput interrogation of a given locus⁴⁵. LP cell lines can be intensively verified following CRISPR/Cas9 expression to ensure the absence of undesired rearrangements or other off-target events, while subsequent Cre expression for payload delivery is expected to be less mutagenic¹².

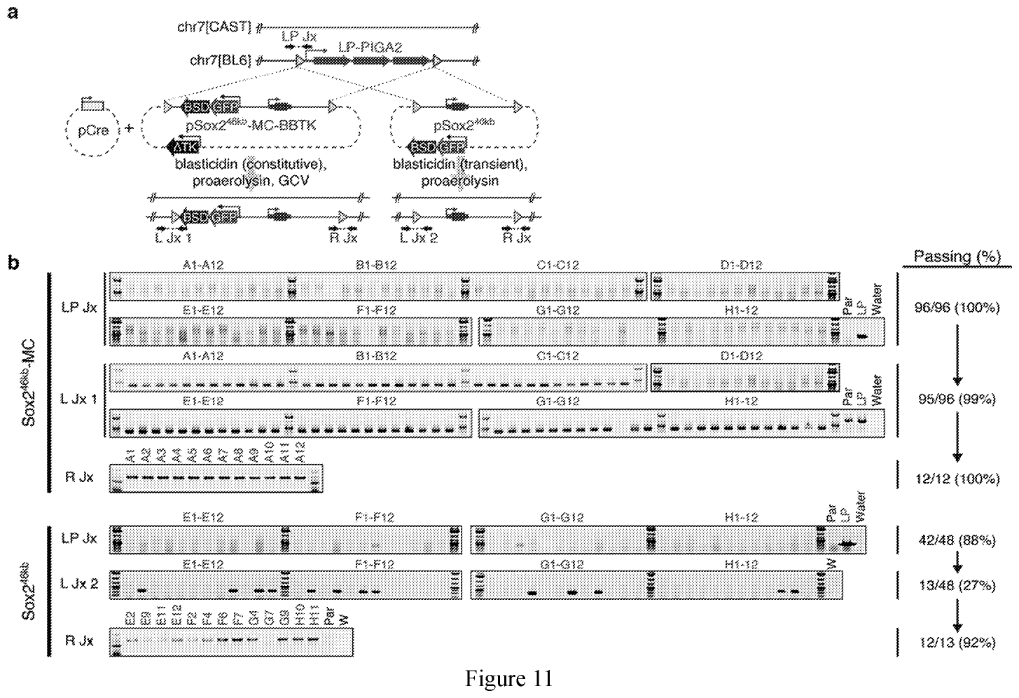

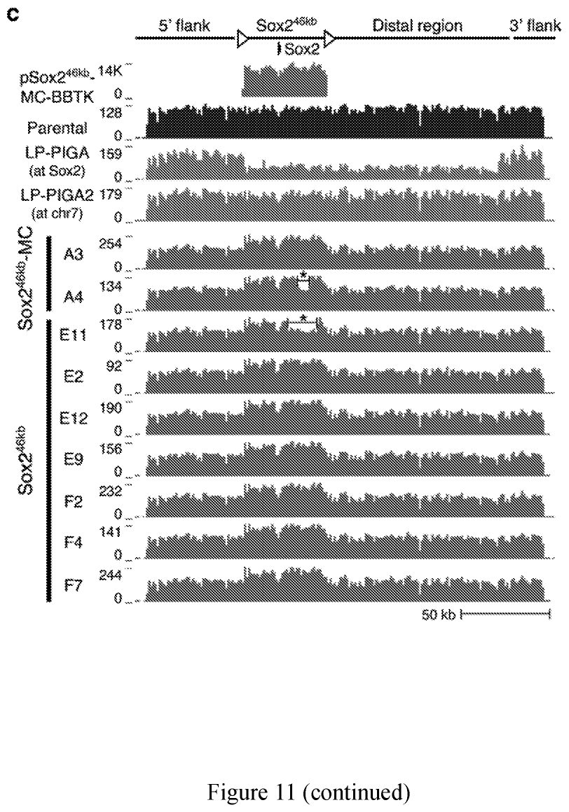

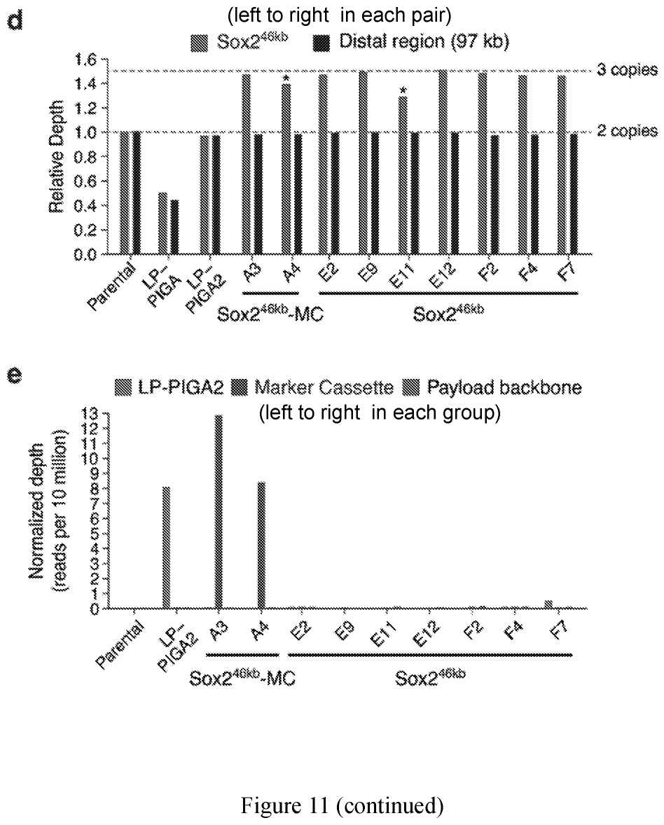

The cell engineering approach is designed to scale rapidly across multiple loci and cell lines. While we have demonstrated Big-IN in both mouse and human ESCs, it is possible that engineering other mammalian cell lines with LPs may require optimization. Indeed, we note the success of the LP-expressed Cre^{ERT2} strategy in H1 hESCs but not in mESCs. We have shown that the selection and delivery methods described herein can be redeployed in a modular fashion to overcome challenges associated with different cell types and loci. For example, the LP can employ either HSV1-ΔTK or hmPIGA as a counterselectable marker, with the former suffering from a bystander effect, and the latter requiring prior engineering to inactivate the endogenous PIGA. Similarly, inclusion of a positive selection marker on the payload augments delivery efficiency, while its placement in the payload backbone enables scarless integration (FIG. 11b). While quantitative comparison of the efficiency of the Big-IN deliveries described herein is affected by technical differences and the need to replate rapidly growing ESCs, the disclosure includes improvements that enhance overall efficiency and its application to diverse cellular contexts.

The described verification strategy is tailored to enable early verification of engineering outcomes. For example, the use of locally generated bait in our Capture-Seq pipeline circumvents the cost and delay of commercially synthesized bait pools. Additionally, bamintersect works with standard libraries generated from genomic DNA, unlike specialized ligation-mediated approaches, and uses standard reference coordinates rather than custom assemblies for each delivery. We demonstrate the value of our pipeline through detection of internal duplications and deletions in integrated payloads that would have been difficult to detect using PCR screening.

The efficiency of Big-IN for integration of large DNA constructs suggests that it can also support integration of complex libraries for saturation mutagenesis of shorter elements⁴⁶, and eventually, analysis of libraries of large constructs in a pooled format. When combined with the rapidly evolving big DNA synthesis field^{14,25}, the disclosure includes use of Big-IN to obtain designer-like control over mammalian genomes and facilitate a synthetic approach to genome biology.

### Example 1

### Engineering the HPRT1 Locus in Human ESCs

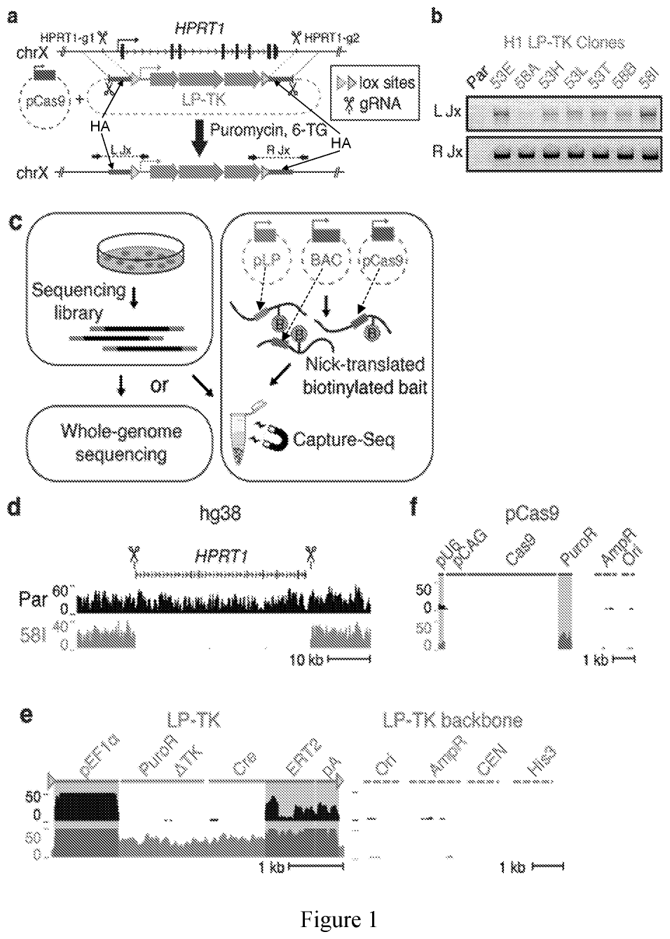

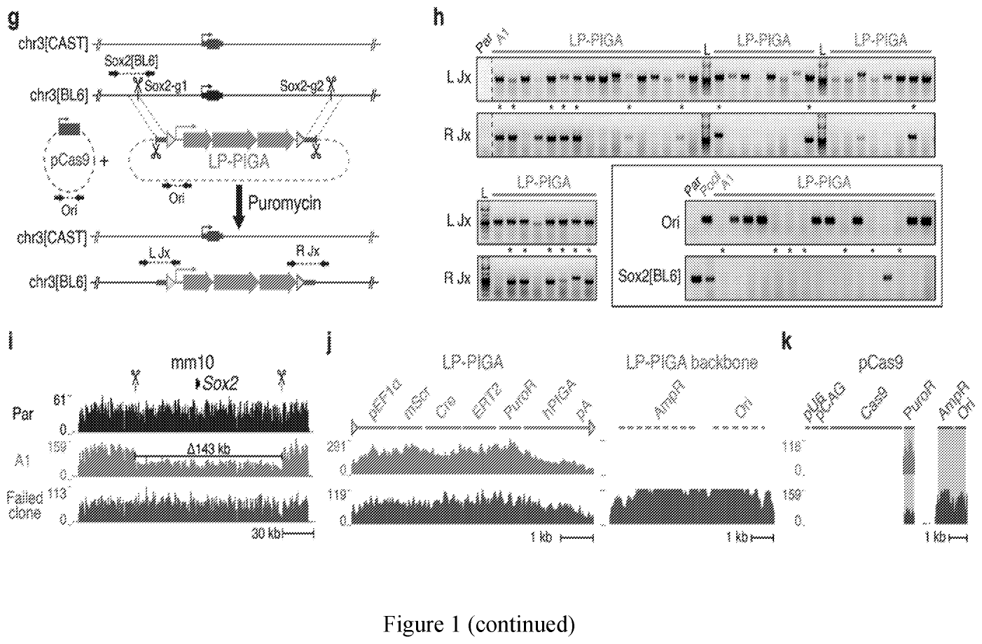

To enable repeated, precise, and efficient delivery of large DNAs to a given locus, we employed a two-stage approach that first targets a short landing pad (LP) to replace a genomic locus of interest using CRISPR-Cas9-mediated Homology Directed Repair (HDR) (FIG. 1a). A plasmid (pLP-TK) was engineered to include the human EF1α promoter (pEF1α) to drive ubiquitous expression of a single open reading frame (ORF) comprising a puromycin-resistance gene (PuroR) fused to a truncated Herpes simplex virus thymidine kinase (HSV1-ΔTK) gene 26 and a Cre^{ERT2} gene²⁷, separated by a P2A peptide²⁸. Interposed between the LP ORF and the vector backbone are heterotypic loxM (lox 2272) and loxP sites to permit subsequent RMCE. The lox sites are flanked by homology arms (HAs) corresponding to the genomic sequences flanking gRNA target sites at the targeted genomic locus. To facilitate clearance of the transiently-transfected plasmid by inducing its linearization in vivo, the same gRNA target sequences and protospacer adjacent motifs (PAM) were cloned into the vector backbone just outside the HAs.

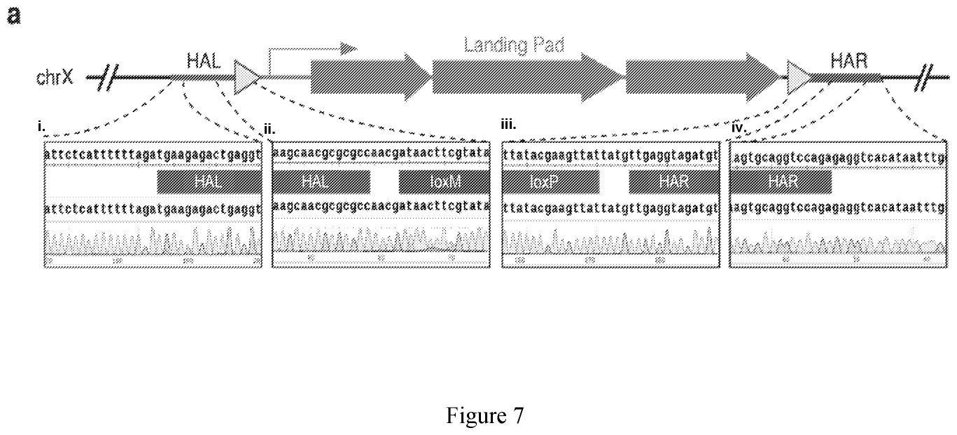

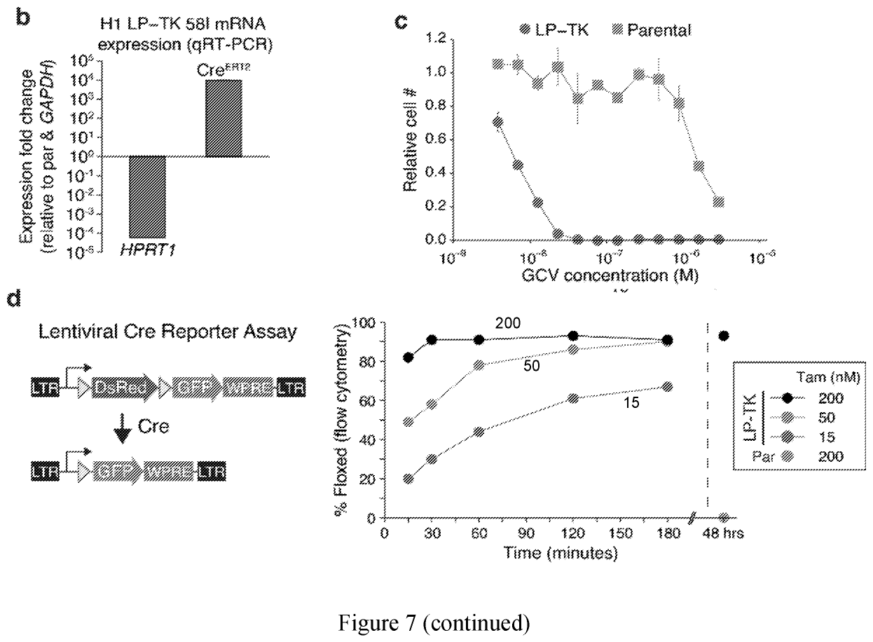

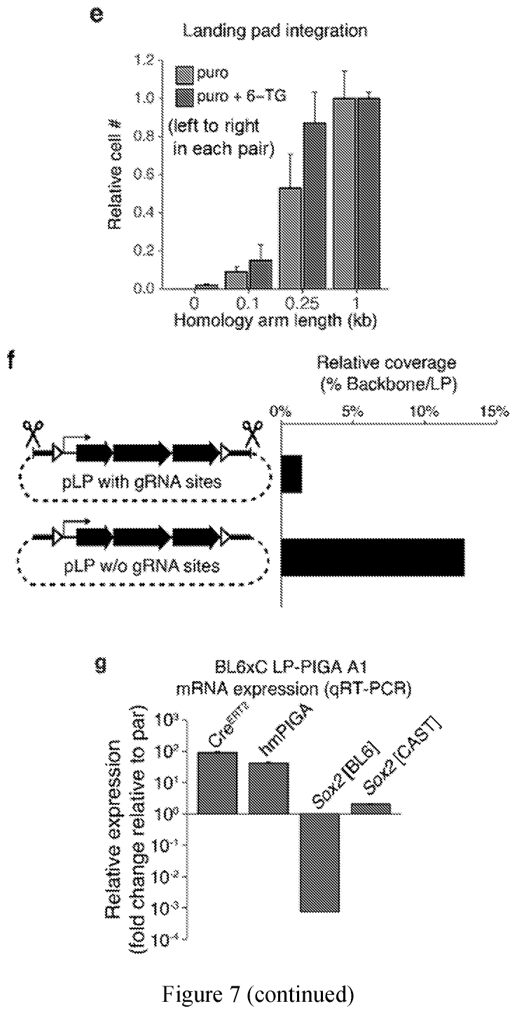

For LP integration, we first targeted the X-linked HPRT1 locus to permit counterselection with the cytotoxic antimetabolite 6-Thioguanine (6-TG)²⁹. H1 male human embryonic stem cells (hESCs), which harbor a single copy of HPRT1, were co-transfected with pLP-TK and pCas9 plasmids³⁰ expressing gRNAs targeting a 42 kb region including the HPRT1 gene for replacement. Cells were sequentially treated with 6-TG and puromycin to select for HPRT1 loss and LP-TK gain, followed by clonal isolation. Correct LP-TK integration was verified by PCR genotyping using primers targeting the novel junctions between LP-TK and the genomic sequences beyond the HAs (FIG. 1b). A candidate clone (581) was selected for further validation. Junction PCR amplicons were subjected to Sanger sequencing, to verify correct LP-TK integration at basepair resolution (FIG. 7a). Quantitative real-time PCR (qRT-PCR) confirmed loss of HPRT1 mRNA expression and gain of Cre^{ERT2} expression (FIG. 7b). Robust cytotoxic activity of HSV1-ΔTK following ganciclovir (GCV) treatment was validated in a kill-curve assay FIG. 7c). We also developed a lentiviral reporter assay for Cre activity, which indicated that Cre^{ERT2} is rapidly and efficiently activated by tamoxifen FIG. 7d). Thus, the function of all three components of the LP ORF was verified.

To facilitate comprehensive genomic verification of multistep cellular engineering with these complex constructs, we developed a modular next-generation sequencing (NGS) analysis approach, which independently maps short reads to both reference genomes (hg38 and mm10) and custom references for each engineering construct. We further developed a custom capture sequencing (Capture-Seq) approach based on nick translation for rapid, flexible, and cost-effective generation of biotinylated bait for hybridization capture to efficiently verify correct engineering of screened clones (FIG. 1c). Using this mapping pipeline, whole-genome sequencing (WGS) of clone 581 verified loss of the targeted HPRT1 locus, gain of LP-TK, and absence of LP-TK backbone and pCas9 (FIG. 1d-f).

Integration relied on 1 kb HAs to correctly target the LP, but HA length reduces the efficiency of PCR genotyping from genomic DNA (FIG. 1b) and impedes the mapping of short sequencing reads that definitively span the LP-HA-genome junctions. Therefore, we measured relative integration efficiency with shorter HAs. We integrated a series of pLP-TK plasmids with varying HA lengths and estimated on-target integration as the relative number of cells surviving puromycin and 6-TG selection, revealing that efficient integration could be performed with HAs as short as 100 bp (FIG. 7e), facilitating subsequent sequence-based mapping of integration sites.

We also assessed the efficacy of our in vivo linearization strategy to reduce off-target integration of transiently-transfected plasmids. We designed two pLP-TK plasmids differing only in the presence of the LP-flanking gRNA sites required for in vivo linearization, targeted them to HPRT1, selected for correct integrants with puromycin and 6-TG, and subjected the pool of cells to Capture-Seq. We found that the relative coverage depth of the LP backbone was lower for the in vivo-linearized pLP-TK (FIG. 7f), possibly due to enhanced HDR efficiency 31 and/or reduced plasmid half-life (which was evident from shortened transient puromycin resistance of the transfected cells).

### Example 2

### Allele-Specific Engineering of the Murine Sox2 Locus

To develop an approach for allele-specific engineering of diploid loci, we employed C57BL6/6J x CAST/EiJ (BL6xCAST or BL6xC) F1 hybrid mouse ESC cells (mESCs) 32, the genome of which harbors heterozygous point variants every 140 bp on average 33. We targeted the Sox2 locus, which encodes a master transcription factor essential for regulation of pluripotency and differentiation^{34,35}. We designed gRNAs targeting the flanks of a 143 kb genomic region that includes the Sox2 coding sequence, promoter, long distance regulatory regions, and several non-coding genes^{35,36}. These designed gRNAs target BL6-specific PAMs to facilitate allele-specific engineering. We constructed pLP-PIGA with a LP including heterotypic loxM/loxP sites and flanked by short homology arms and gRNA target sites (FIG. 1g). LP-PIGA ORF includes 4 components, separated by 3 mutually-recoded P2A peptides, namely mScarlet³⁷, cre^{ERT2}, PuroR, and a human Phosphatidylinositol Glycan Anchor Biosynthesis Class A minigene (human mini PIGA, hmPIGA), which is used for counterselecting LP-PIGA cells following pre-engineering to delete the endogenous Piga gene, as explained below.

We transfected pLP-PIGA and pCas9 plasmids into BL6xCAST mESCs, selected cells with puromycin and isolated clones. Of 40 clones screened using PCR genotyping, 16 (40%) contained both novel junctions (FIG. 1h). Passing clones were further screened with primers to detect Ori (common to multiple vector backbones), which eliminated 8 Ori-positive clones (50%), likely resulting from retention or off-target integration of LP-PIGA backbone or pCas9. We confirmed the allele-specific loss of Sox2 in 15 (94%) of the 16 clones using a BL6 allele-specific primer harboring 4 mismatched bps relative to the CAST allele (Supplementary Table 5).

A successful LP-PIGA integration (clone A1) and a failed LP-PIGA clone were subjected to Capture-Seq using bait generated from a BAC covering the Sox2 region, and the pLP-PIGA and pCas9 plasmids. Inspection of coverage depth at the 143 kb Sox2 genomic locus revealed a 50% reduction for clone A1 compared with parental mESCs or the failed clones (FIG. 1i), as expected for complete loss of the targeted BL6 allele. Clone A1 also showed specific gain of LP-PIGA with no coverage of the LP-PIGA backbone or pCas9, whereas the failed clone showed clear presence of the LP-PIGA backbone (FIG. 1j-k). Finally, qRT-PCR analysis verified the expression of LP-PIGA components and the BL6 allele-specific loss of Sox2 expression in clone A1 (FIG. 7g), which was chosen for future payload deliveries. In summary, we have demonstrated an efficient strategy for allele-specific LP integration and a comprehensive pipeline for verification of correctly engineered cells.

### Example 3

### Efficient Counterselection for Delivery

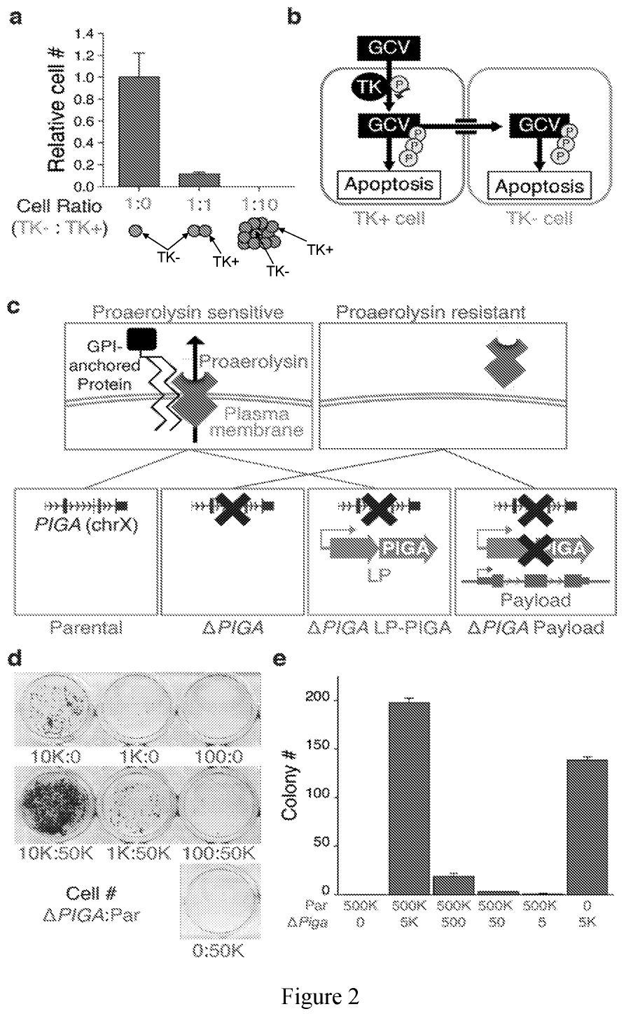

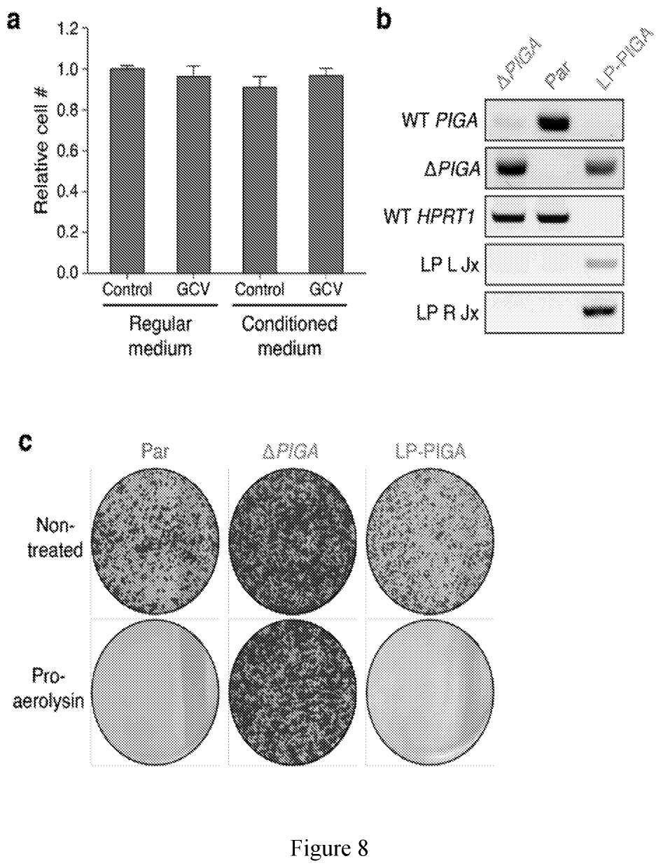

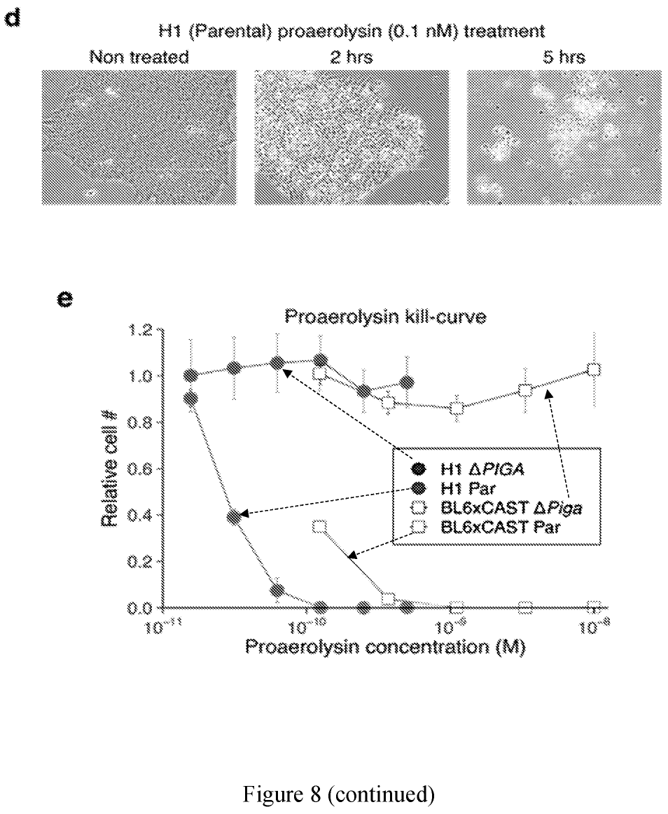

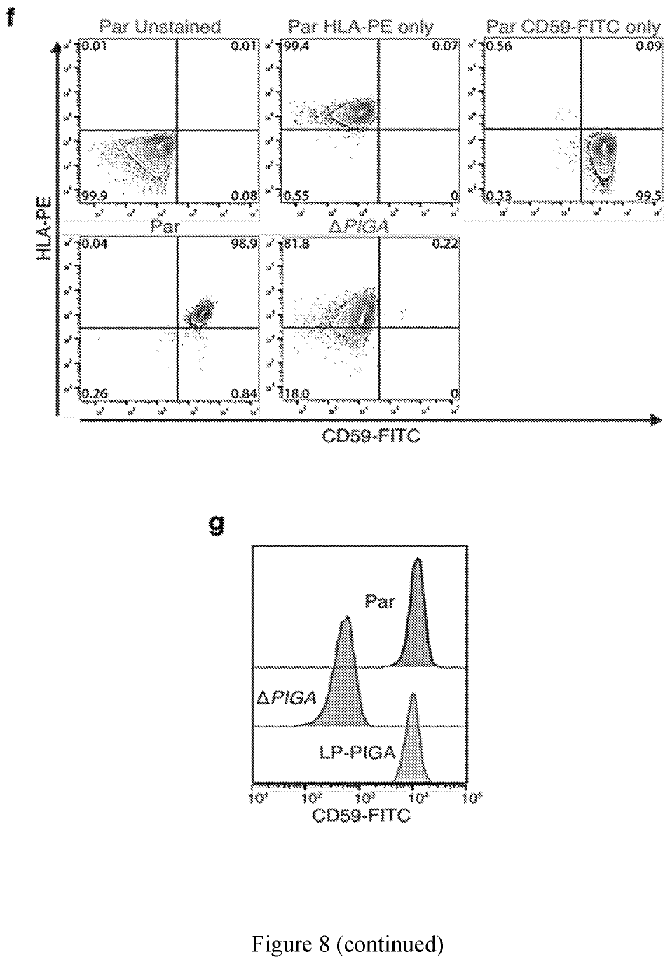

Delivery of large DNA through cassette exchange is an infrequent event, requiring selection to obtain practical efficiency. The HSV1-TK gene is a widely used counterselectable marker that renders cells sensitive to GCV by converting it to the toxic metabolite GCV-triphosphate (GCV-TP), which inhibits DNA synthesis and leads to cell death³⁸. To test the efficacy of TK/GCV counterselection in H1 hESCs, we mixed TK-negative and TK-positive (LP-TK) cells at different ratios and treated these co-cultures with GCV. More than 80% of the TK-negative cells died when mixed at a 1:1 ratio with TK-positive cells, and all died when mixed at a 1:10 ratio (FIG. 2a). Indeed, it is known that GCV-TP can diffuse from TK-positive cells to TK-negative cells via gap junctions^{39,40}. The resulting bystander cell death in TK-negative cells limits the ability to recover rare events (FIG. 2b, FIG. 8a).

Therefore we tested an alternative counterselection strategy that relies on the X-linked PIGA (phosphatidylinositol glycan class A) gene, which encodes an enzyme crucial for the biosynthesis of glycosylphosphatidylinositol (GPI) anchors⁴¹ and renders cells sensitive to proaerolysin, a bacterial prototoxin. Proaerolysin perforates the plasma membrane upon binding to GPI anchors on the cell surface, resulting in rapid cell death⁴². Further, PIGA activity can be quantitatively monitored by measuring levels of CD59, a broadly expressed membrane-linked GPI-anchored protein⁴³. Deletion of PIGA can be selected for with proaerolysin after a short period to allow for loss of PIGA protein and subsequent loss of GPI-anchored proteins from the cell surface⁴⁴.

While proaerolysin efficiently killed parental H1 hESCs, ΔPIGA cells, in which the PIGA gene was deleted using CRISPR/Cas9 (see Methods), were entirely resistant (FIG. 7b-e). Integration of a landing pad expressing a human mini PIGA gene (hmPIGA) to the HPRT1 locus resensitized H1 ΔPIGA hESCs to proaerolysin and restored CD59 expression (FIG. 7c, FIG. 7g). Importantly, rare ΔPIGA H1 hESCs were efficiently isolated when co-cultured with parental H1 cells by applying proaerolysin selection (FIG. 2d). Similarly, the Piga/proaerolysin counterselection strategy was used to efficiently isolate rare mESCs (FIG. 2e). This suggested that LP-expressed hmPIGA permits negative selection of LP-PIGA cells to effectively enrich for correct delivery events (FIG. 2c).

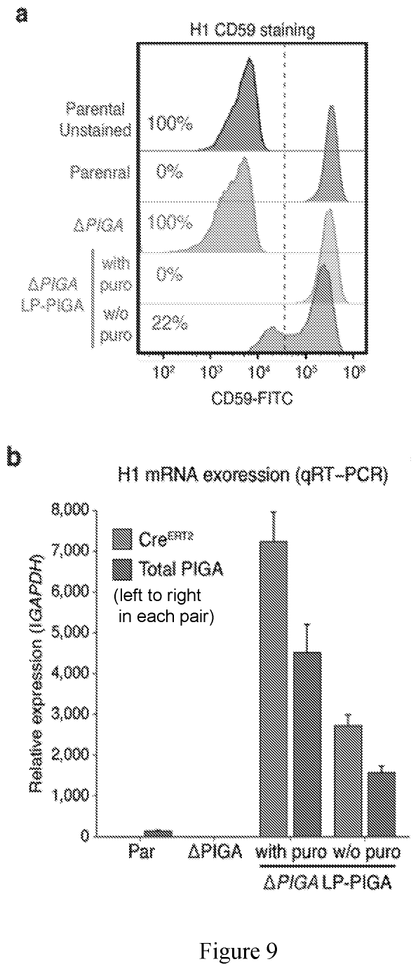

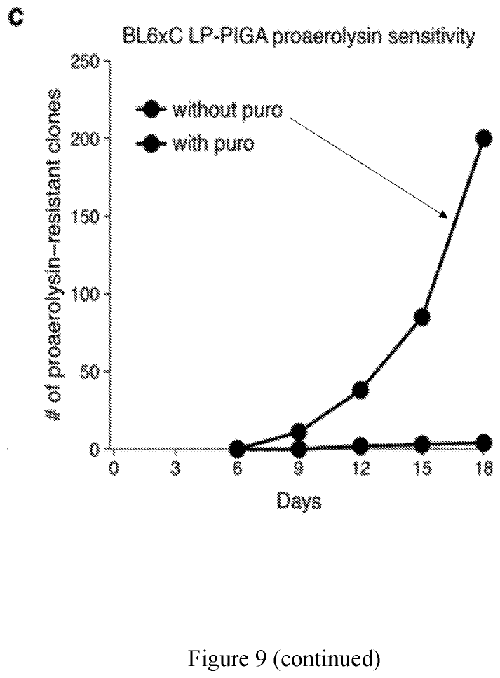

Recovery of rare events where a payload replaces the LP requires that expression of hmPIGA is stably maintained following withdrawal of positive selection. However, while nearly all H1 LP-PIGA cells maintained high CD59 levels in the presence of puromycin, a substantial proportion of cells spontaneously lost CD59 FIG. 9a) and showed reduced PIGA expression (FIG. 9b) following puromycin withdrawal. BL6xCAST LP-PIGA mESCs also spontaneously acquired proaerolysin resistance in the absence of puromycin (FIG. 9c). Thus, any counterselection-based delivery scheme must avoid a potentially high background of false positive cells from LP silencing.

### Example 4

### Efficient Delivery to Human and Mouse ESCs

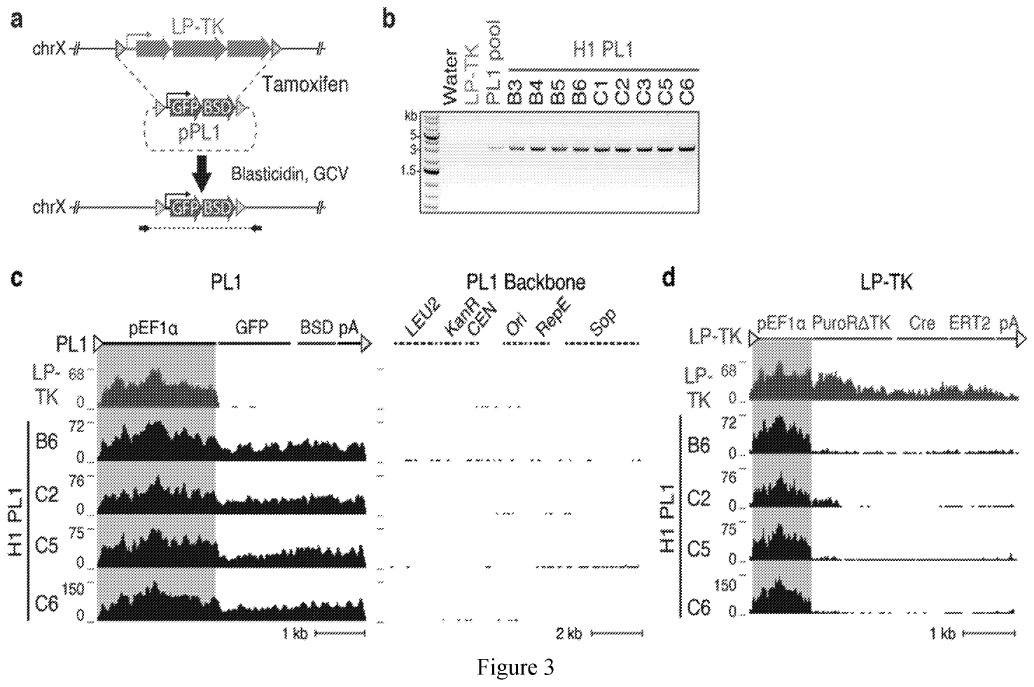

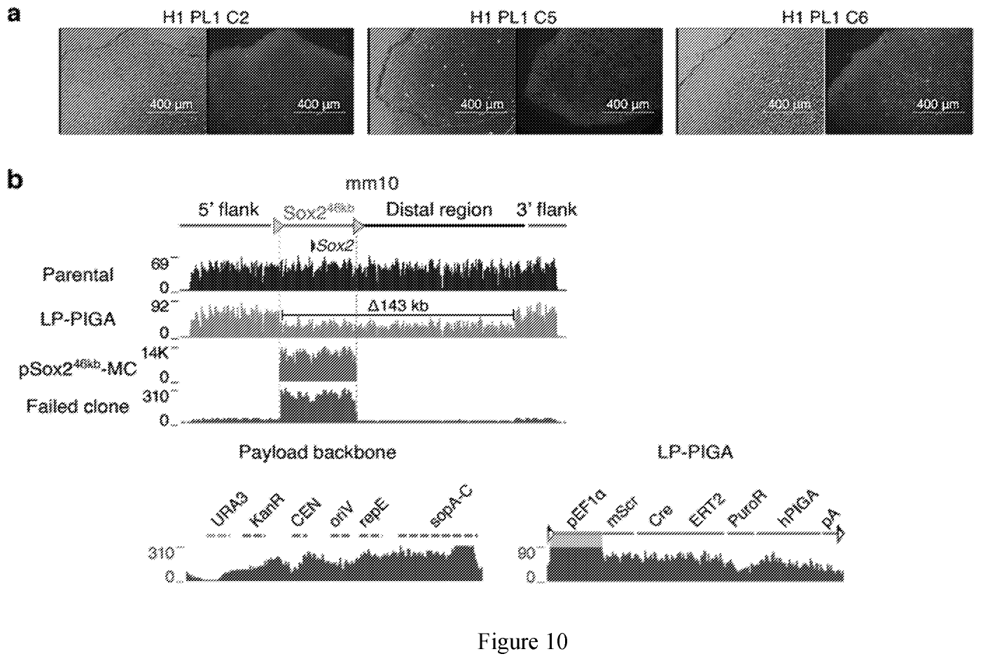

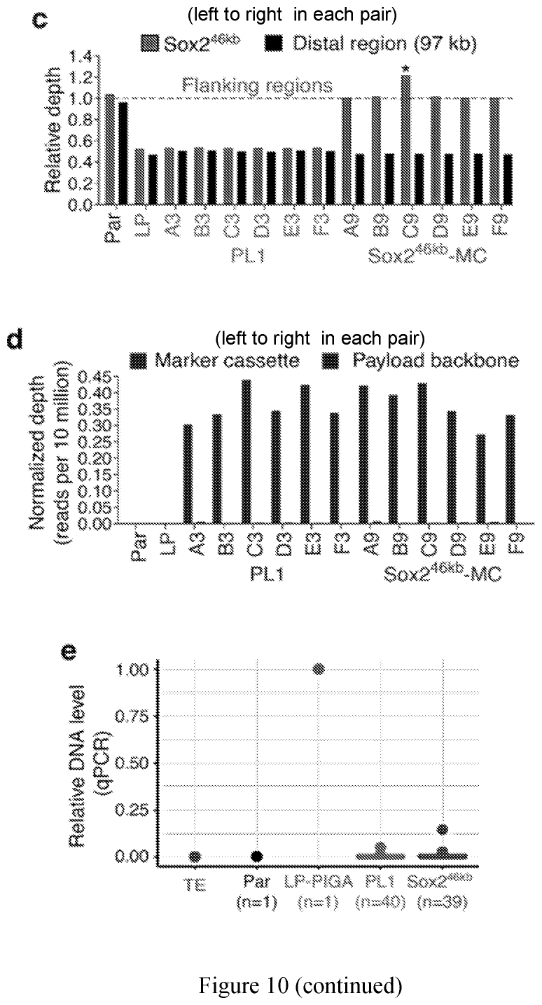

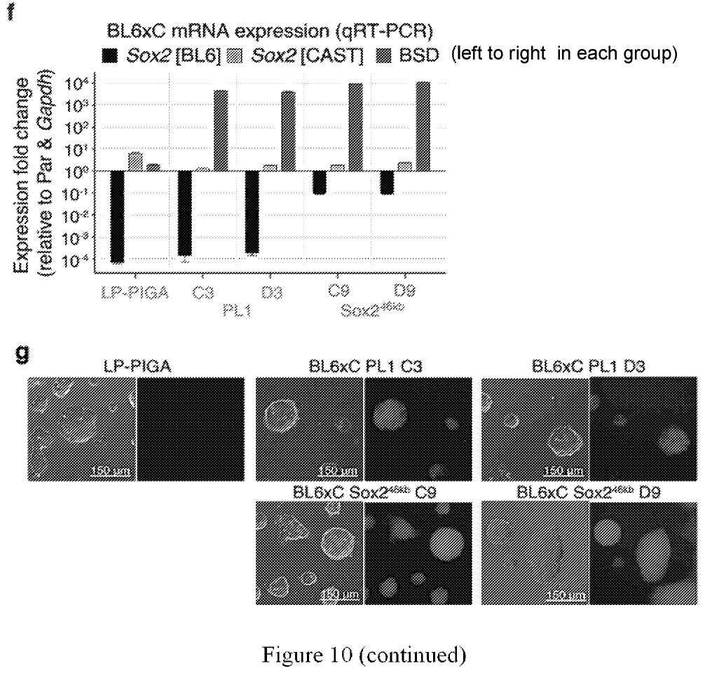

To demonstrate a counterselection-based approach to isolation of successful RMCE events, we designed a minimal 2.7 kb payload (PL1), comprising an pEF1α-driven GFP-T2A-BSD ORF flanked by loxM and loxP sites (FIG. 3a). H1 LP-TK cells were transfected with a PL1-harboring plasmid (pPL1) and LP-derived Cre^{ERT2} activity was induced with tamoxifen. Cells were selected with blasticidin to enrich for PL1-expressing cells, followed by GCV counterselection of TK-expressing cells. PCR genotyping of isolated clones showed a 100% rate of replacement of LP-TK with PL1 (FIG. 3b). Capture-Seq analysis of 4 selected clones confirmed the presence of PL1, the absence of any plasmid backbone, and the loss of LP-TK (FIG. 3c-d). The integrated PL1 was transcriptionally active, as evident from GFP expression (FIG. 10a).

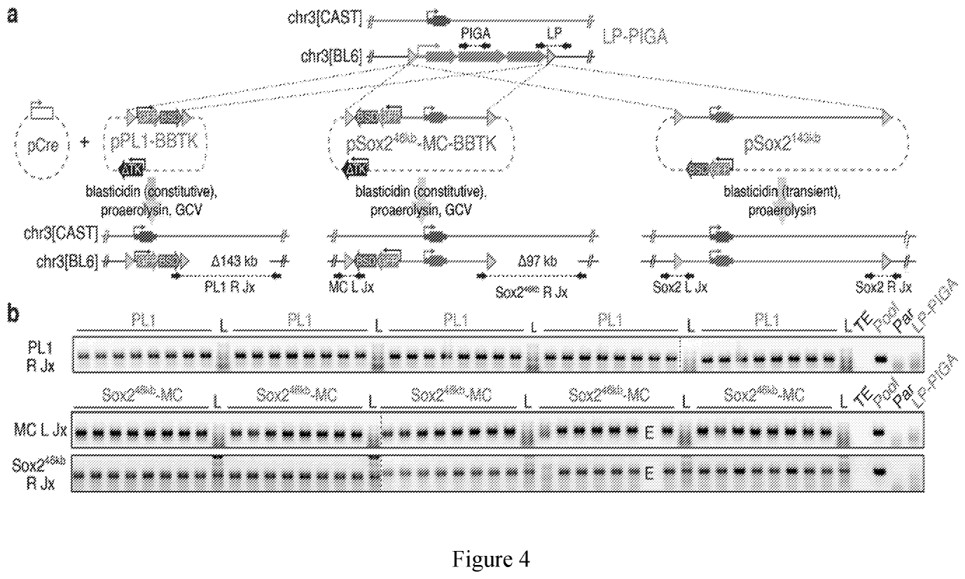

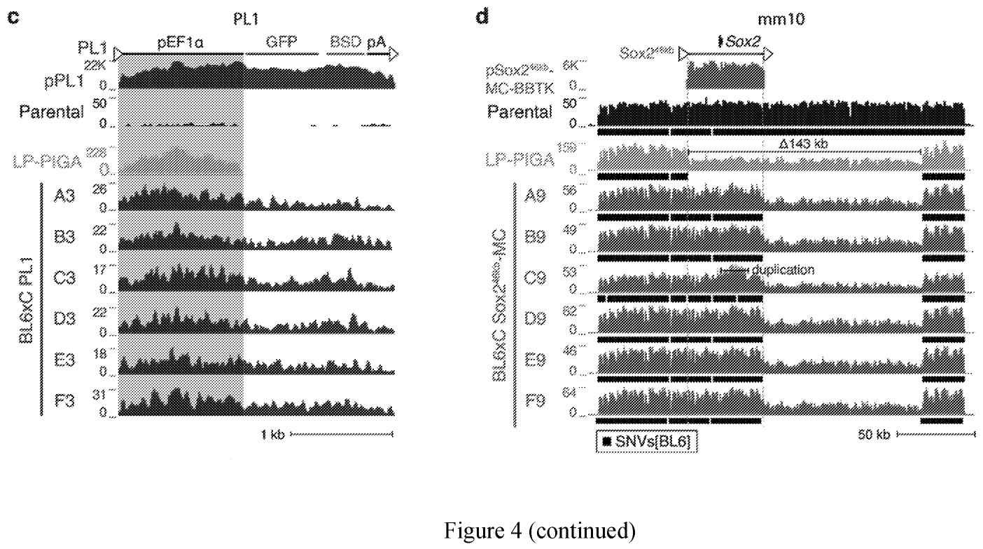

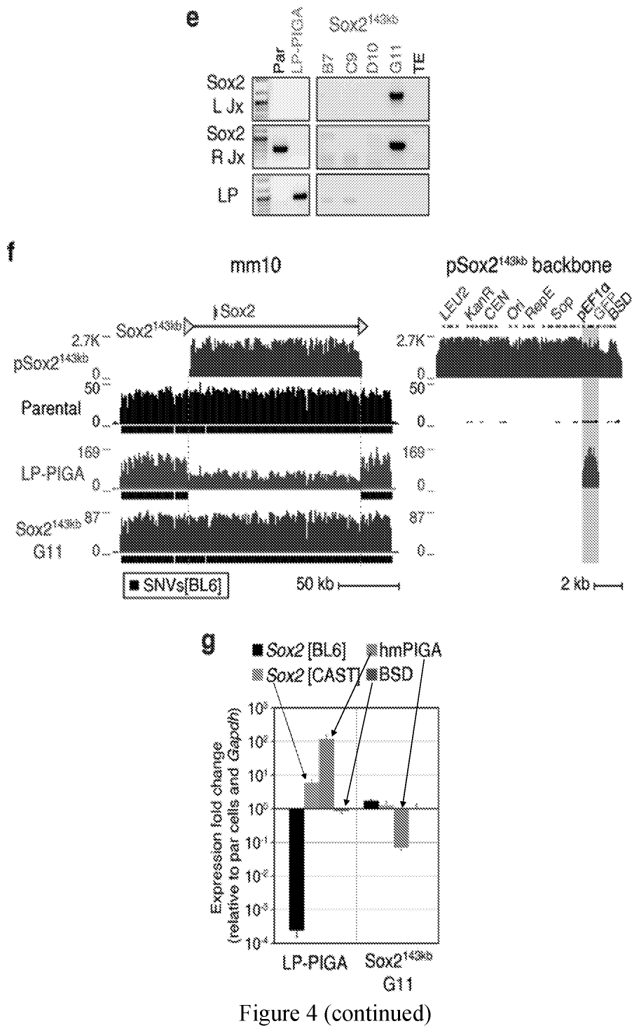

We attempted to apply a similar strategy for delivery to LP-PIGA mESCs. However, all clones that survived blasticidin and proaerolysin selection manifested multicopy gain of payload and vector backbone without LP-PIGA loss (FIG. 10b). We transiently augmented Cre activity through co-transfection of a Cre expression plasmid (pCAG-Cre). Additionally we cloned a ΔTK expression cassette (BBTK) into the payload backbone to permit GCV counterselection against off-target integrants. Co-transfection of pPL1-BBTK and pCAG-iCre readily resulted in efficient PL1 integration. To assess efficiency of larger payloads, pSox2^{46kb}-MC-BBTK was constructed including a 46 kb region of the Sox2 locus and containing a marker cassette to enable positive selection (FIG. 4a). Upon delivery and selection, PCR genotyping verified that 99% of clones harbored correct payload integration (FIG. 4b). Six PCR-validated clones of each payload type were then chosen for Capture-Seq analysis. Mapping sequencing reads to the PL1 sequence or mouse genome revealed that all clones had complete coverage of the delivered payload (FIG. 4c). Coverage depth was restored to parental levels over the genomic region corresponding to Sox2^{46kb}, while the remaining 97 kb of the Sox2 deletion was unaffected (FIG. 4d and FIG. 10c). Analysis of known CAST single nucleotide variants (SNVs) further confirmed re-introduction of BL6 alleles. There was no evidence for the gain of the payload backbone in any of the clones analyzed (FIG. 10d), and all 79 clones lost LP-PIGA (FIG. 10e). Selected PL1 and Sox2^{46kb}-MC cells both expressed the payload-derived BSD, while Sox2^{46kb}-MC clones also partially restored the expression of the BL6 allele of Sox2 (FIG. 10g). In addition, both cell types showed expression of payload-derived GFP (FIG. 10f).

This approach leaves a BSD-GFP transcriptional unit (TU) integrated with the payload, which might affect the activity of nearby genes or regulatory elements. To develop an alternate architecture and selection strategy for scarless delivery, we constructed pSox2^{143kb}, which harbors the entire 143 kb Sox2 BL6 allele replaced by LP-PIGA, and in which the BSD-GFP TU is relocated on the backbone outside the lox sites (FIG. 4a). We delivered pSox2^{143kb} to LP-PIGA mESCs together with pCAG-iCre, which encodes a codon-optimized Cre recombinase, and selected cells transiently with blasticidin to enrich for payload-transfected cells, followed by proaerolysin selection to eliminate LP-PIGA cells. PCR genotyping identified 4 clones that lost LP-PIGA, one of which (G11) was positive for the newly-formed BL6 allele genomic junctions (FIG. 4e). Capture-Seq analysis verified the restoration of the entire 143 kb BL6 allele in clone G11, without gain of the payload backbone (FIG. 4f). Finally, qRT-PCR analysis confirmed that the expression of the BL6 allele of Sox2 was completely restored, and expression of hmPIGA and BSD was undetectable (FIG. 4g).

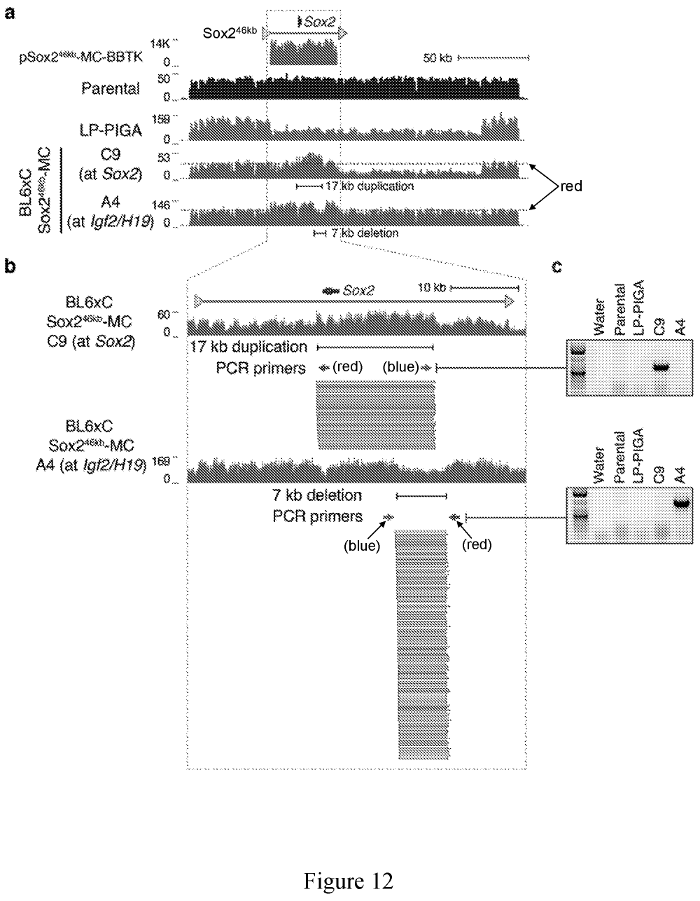

To demonstrate the flexibility of Big-IN for delivery of payloads to additional loci, LP-PIGA2 was integrated into chromosome 7 of BL6xCAST mESCs, replacing a 157 kb region of the Igf2/H19 locus (FIG. 11a). We transfected these cells with pCAG-iCre and either the non-scarless payload pSox2^{46kb}-MC-BBTK or the scarless pSox2^{46kb} payload. Following stable positive selection with blasticidin and negative selection with proaerolysin and GCV, 95/96 (99%) of Sox2^{46kb}-MC clones were verified by PCR for the loss of LP-PIGA2 and the gain of the novel left payload junction (FIG. 11b). Conversely, following transient blasticidin and proaerolysin selection, 12/48 (25%) Sox2^{46kb} clones were similarly verified. Further verification of selected clones confirmed the presence of the right payload junction for 24/25 clones and the absence of pCAG-iCre in all clones. Capture-Seq analysis of chosen clones confirmed specific payload gain without detectable payload backbone and complete loss of LP-PIGA2 (FIG. 11c-d). Notably, Capture-Seq analysis also identified clones with defects not easily detectable through PCR genotyping, including an internal payload duplication in BL6xCAST Sox2^{46kb}-MC clone C9 and an internal payload deletion in BL6xCAST Sox2^{46kb}-MC clone A4 (FIG. 12).

### Example 5

### Genomic Screening of On- and Off-Target Integrations

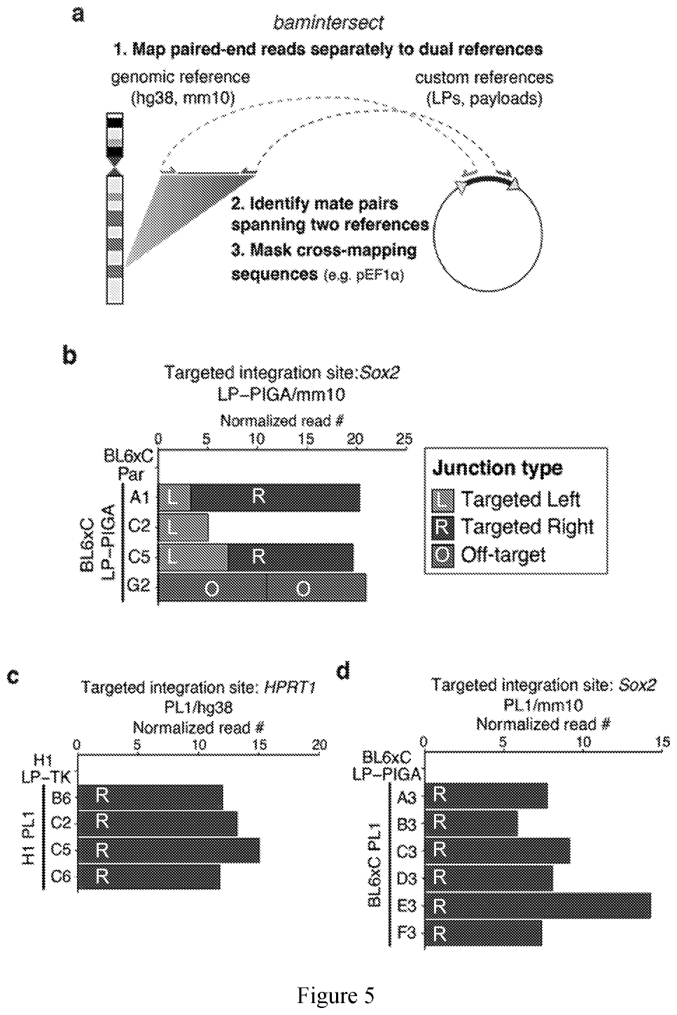

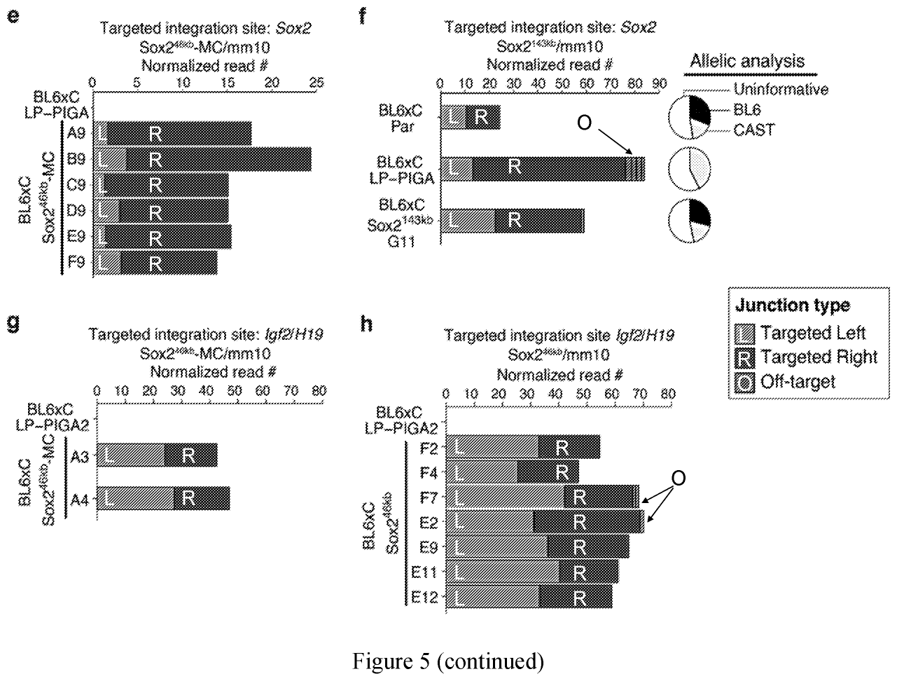

In order to screen genomic data for on- and off-target integration events, we developed bamintersect. Bamintersect leverages our modular mapping approach to analyze reads mapped separately to two reference genomes and detect read pairs indicative of a junction (FIG. 5a, Methods). Nearby reads are clustered and thresholded, and masked for uninformative regions. We applied bamintersect to confirm LP integration and payload delivery for the genomic engineering events described herein, the majority of which were verified by identifying multiple reads supporting the novel junctions between the integrated sequence and its flanks (FIG. 5). Specifically, for LP-PIGA integration at Sox2, two out of the four analyzed clones (A1 and C5) were validated for correct integration, whereas one clone (C2) was validated only for the left junction, and an additional clone (G2) demonstrated off-target LP integration at chromosome 1 (FIG. 5b). In contrast, all analyzed payload clones were verified as correct (FIG. 5c-h).

Of note, several novel engineered junctions were impossible to confirm using bamintersect due to technical reasons, including LP-TK integration at HPRT1, for which the 1 kb homology arms precluded mapping reads that span the junction between LP-TK and hg38, as well as PL1 deliveries to both HPRT1 and Sox2, for which the left junction is nearly identical to that of the replaced LP.

For Sox2^{143kb} deliveries, the newly-formed payload-genome junctions are nearly identical to the original sequences in parental cells (deleted in LP-PIGA mESCs), as well as to the existing regions in the CAST allele. We therefore categorized bamintersect read pairs that overlap with BL6xCAST variants according to their genotypes, revealing that while LP-PIGA mESCs junctions are depleted of BL6 reads, these reads are restored in Sox2^{143kb} clone G11 mESCs (FIG. 5f), validating the correctly restored BL6 allele.

Combined, these results support the utility of bamintersect as a sensitive, scalable and unbiased tool for detection of on and off-target integration events.

### Example 6

### Methods

### Cloning and Isolation of DNA Constructs

Primers used for cloning are listed in Supplementary Table 1. pLP-TK (pLP050/pJML0050) was assembled by a combination of overlap PCR, Gibson assembly of intermediate fragments, and Golden Gate cloning. The backbone was assembled from PCR-amplified fragments: The HIS3 transcriptional unit (TU) fragment was amplified as two overlapping parts to remove an internal BbsI site from pRS413 (ATCC, 87518) using primers oJML0069+oJML0056 and oJML0057+oJML0058. The Ori-AmpR-CEN/ARS fragment was amplified as two overlapping parts to remove an internal BsaI site from pRS413 using primers oJML0053+oJML0068 and oJML0067+oJML0070. These parts were combined with a synthetic sequence containing Golden Gate compatible cloning sites for adding homology arms, and cloned by Gibson assembly. LP-TK, consisting of loxM, pEF1α-driven PuroRΔTK-P2A-Cre^{ERT2} coding sequence, EIF1 polyadenylation signal (EIF1 pA), and a loxP site, was assembled largely by overlap PCR followed by Golden Gate assembly into the above mentioned backbone: The PuroRΔTK-P2A-Cre^{ERT2} fragment was built by overlap PCR of PuroR using oJML0126+oJML0144 and pJML0010 as a template, ΔTK-P2A using oJML0137+oJML0129/oJML0138 with pSP0130 as a template, P2A-Cre^{ERT2} with oJML0130/oJML0139+oJML0131 and oJML0132+oJML0133/oJML0134 and pBabe-Puro Cre ERT244 as a template, and EIF1 pA with oJML0135+oJML0136 and pJTR0085 as a template. The assembled coding sequence was cloned into the backbone by BbsI-mediated Golden Gate assembly. pEF1α was amplified from pSP0044 with primers oJML0145+oJML0146 and cloned into the LP-containing vector by BsmBI-mediated Golden Gate assembly.

pLP-PIGA (pLP140/pJML0140) was cloned using BbsI-mediated Golden Gate assembly of a synthetic LP fragment, consisting of loxM/loxP-flanked pEF1α-driven mScarlet-P2A-Cre^{ERT2}-P2A-PuroR-hmPIGA coding sequence, and an EIF1 pA, into a minimal ‘entry vector’. The entry vector (pJML0100) was modified from a minimal bacterial backbone (pYTK095, Addgene plasmid #65202) by inserting a BbsI Golden Gate-compatible entry sequence at the NotI site.

pLP-PIGA2 (pLP300/pRO_009) was constructed from a synthetic plasmid that included the following LP region components: loxM-pEF1α-PuroR-P2A-hmPIGA-P2A-mScarlet-EIF1 pA-loxP (where the P2A sequences are mutually recoded). A ΔTK synthetic transcriptional unit consisting of a human PGK1 promoter, an HSV1-ΔTK gene, and an SV40 polyadenylation signal was cloned into the SbfI site in the pLP backbone and a clone in which the two TUs are facing opposite ways was identified by PCR.

To facilitate targeting of LPs to specific genomic loci, homology arms (HAs) corresponding to the genomic sequence flanking the Cas9 cut sites were amplified from either mammalian genomic DNA or from a BAC corresponding to the engineered region. Homology arms were cloned distally to the LoxM and LoxP site in the LP using a BsaI Golden Gate assembly reaction. Primers used to amplify homology arms are listed in Supplementary Table 2.

pPL1 was assembled in yeasto⁴⁷ from 3 linear DNA fragments, each encoding ≥40 bp terminal sequence homology with its adjacent fragments. These fragments included a BsaI-digested pLM1050 yeast/E. coli shuttle vector, a pEF1α-GFP cassette amplified using PCR primers oRB_061+oRB_063 from pSP0108 and a T2A-BSD-bGHpA (bGHpA, bovine growth hormone polyadenylation signal) cassette amplified using PCR primers oRB_062+oRB_064 from pSP0172.

pPL1-BBTK (pJML0206) was constructed from pPL1 in yeasto. pPL1 was linearized using recombinant Cas9 (New England Biolabs M0386) and a synthetic tracrRNA/crRNA (TTGCGCACGGTTATGTGGAC) (SEQ ID NO:1) duplex (Integrated DNA Technologies, IDT). A ΔTK synthetic transcriptional unit, consisting of a human PGK1 promoter driving the expression of a recoded ΔTK gene and an SV40 polyadenylation signal, was amplified to carry overlapping homology to the Cas9-digested pPL1 backbone. Linear fragments were co-transformed to yeast, and colonies were screened by colony PCR.

pSox2^{46kb} (pLM1113) and pSox2^{143kb} (pLM1120) were constructed in yeasto in a two-step process starting with a BAC that carries the Sox2 locus (BACs and relevant genomic coordinates are listed in Supplementary Table 3). The BAC was subjected to in vitro CRISPR/Cas9 digestion using synthetic gRNAs mSox2-g1 and mSox2-g3 to release a 46 kb segment or mSox2-g1 and mSox2-g2, to release a 143 kb segment. Specifically, synthetic crRNAs and tracrRNA (IDT) were resuspended and mixed at 1 μM each with Duplex Buffer (IDT), heated to 94° C. and slowly cooled to room temperature. Next, 1 μL of duplexed crRNAs/tracrRNA were mixed with 2 μL 10×Cas9 Buffer and 1 μL recombinant Cas9 (New England Biolabs M0386S) in a total volume of 20 incubated for 10 min at room temperature, supplemented with 1 μg BAC DNA and incubated for 2 hours at 37° C., followed by inactivation with 1 μL Proteinase K (Qiagen 19131) for 10 min at room temperature. The digestion products were co-transformed with BsaI-digested assembly vector pLM1110 and terminal linker sequences (250 bp gBlocks, IDT) to enable homologous recombination-dependent assembly.

pSox2^{46kb}-MC (pLM1121) was cloned by digesting pSox2^{46kb} (pLM1113) using I-SceI and assembling in yeasto with a selectable marker cassette containing pEF1α-GFP-T2A-BSD-bGHpA, which was PCR-amplified from pPL1, a BsaI-digested pLM1081 yeast/E. coli shuttle vector and 3 gBlock (IDT) linkers to provide terminal homology between parts.

pSox2^{46kb}-MC-BBTK (pJML0207) was cloned from pSox2^{46kb}-MC (pLM1121) in the same manner as pPL1-BBTK was built from pPL1, using the same guide sequence and ΔTK fragment.

Payload constructs were recovered from yeast and transformed into CopyControl TransforMax EPI300 E. coli cells (Lucigen). A single colony was grown overnight in selective LB medium at 37° C. with shaking and then subcultured 1:100 in 150-300 mL selective LB medium supplemented with CopyControl Induction Solution (Lucigen) and grown for an additional 6-8 hours.

All gRNAs were cloned into pSpCas9(BB)-2A-Puro V2.0 (pCas9) plasmids using BbsI Golden Gate assembly as described³⁰. gRNA sequences and genomic target coordinates are listed in Supplementary Table 4.

The lentiviral Cre reporter construct pLV-lox-dsRed-lox-GFP was cloned by amplifying a loxP-dsRed-loxP-eGFP cassette from pMSCV-loxP-dsRed-loxP-eGFP-Puro-WPRE⁴⁸ (Addgene plasmid #32702) using primers oRB_036+oRB_037, digesting the product with ClaI+NotI and ligating into a ClaI+NotI-digested lentiviral vector pLH1263. The resulting lentivirus encodes a pEF1α-driven loxP-dsRed-loxP-eGFP-WPRE.

Plasmids were isolated using the ZymoPURE II Plasmid Maxiprep Kit (Zymo Research D4203) according to the manufacturer's protocol. BACs and large payloads were isolated using the NucleoBond Xtra BAC kit (Takara Bio 740436).d gRNA design gRNAs were designed using the GuideScan algorithm⁴⁹. For allele-specific LP integration at Sox2 we produced a scored list of potential gRNAs targeting a 261 kb region surrounding Sox2 using the BL6 reference genome sequence. Next, we identified gRNAs for which the corresponding PAM is mutated in the CAST allele, resulting in a list of BL6-specific gRNAs. From this list we selected two high-scoring gRNAs, Sox2-g1 and Sox2-g2, which target a 143 kb genomic region for replacement with the LP. gRNA sequences are listed in Supplementary Table 4.

### Cell Culture

WA01 (H1) human embryonic stem cells (hESCs) were purchased from WiCell. H1 hESCs were initially grown for 2 weeks on plates coated with Matrigel (Corning 354277) in mTeSR medium (Stem Cell Technologies 85850) and subsequently transferred to plates coated with Geltrex (Gibco A1413302) and StemFlex medium (ThermoFisher A3349401) supplemented with 1% Pen-Strep (ThermoFisher 15140122). For routine passaging, cells were dissociated into clumps with Versene (Gibco 15-040-066) and gentle trituration. Wide-orifice pipette tips were used when handling small volumes of cell suspension.

C57BL6/6J x CAST/EiJ (BL6xCAST) clone 4 mESCs32 were used. mESCs were cultured on plates coated with 0.1% gelatin (EMD Millipore ES-006-B) in 80/20 medium comprising 80% 2i medium and 20% mESC medium. 2i medium contained a 1:1 mixture of Advanced DMEM/F12 (ThermoFisher 12634010) and Neurobasal-A (ThermoFisher 10888022) supplemented with 1% N2 Supplement (ThermoFisher 17502048), 2% B27 Supplement (ThermoFisher 17504044), 1% Glutamax (ThermoFisher 35050061), 1% Pen-Strep (ThermoFisher 15140122), 0.1 mM 2-Mercaptoethanol (Sigma M3148), 1250 U/ml LIF (ESGRO ESG11071), 3 μM CHIR99021 (R&D Systems 4423) and 1 PD0325901 (Sigma PZ0162). mESC medium contained Knockout DMEM (ThermoFisher 10829018) supplemented with 15% Fetal Bovine Serum (FBS, BenchMark 100-106), 0.1 mM 2-Mercaptoethanol, 1% Glutamax, 1% MEM Non-Essential Amino Acids (ThermoFisher 11140050), 1% Nucleosides (EMD Millipore ES-008-D), 1% Pen-Strep and 1250 U/ml LIF. HEK-293T cells were cultured in DMEM supplemented with 10% FBS, 1 mM sodium pyruvate (ThermoFisher 11360070), 1% Glutamax and 1% Pen-strep. All cells were grown at 37° C. in a humidified atmosphere of 5% CO2 and passaged on average twice per week.

### Chemicals and Treatments

Puromycin (Sigma P9620) and Blasticidin S (ThermoFisher R21001) were applied as described below. Ganciclovir (GCV, Sigma PHR1593) was dissolved in water and NaOH at pH 12 and adjusted to pH 11 with HCl and water to a final concentration of mg/ml. GCV and Proaerolysin (Aerohead Scientific) concentrations are indicated below. 4-Hydroxytamoxifen (tamoxifen, Sigma T176) was applied at 200 nM, unless indicated otherwise. 6-TG (Sigma A4660) was applied at 30 μM.

### Genome Engineering

Relevant genomic coordinates are listed in Supplementary Table 3.

H1 hESCs were transfected using the Neon Transfection System (ThermoFisher). Cells were treated several hours prior to transfection with StemFlex medium supplemented with 1% RevitaCell Supplement (ThermoFisher A2644501). Cells were washed with PBS, dissociated into a single-cell suspension using TrypLE-Select (ThermoFisher 12563011), which was neutralized with StemFlex medium, spun down at 200 rcf for 3 min, supernatant aspirated and cells resuspended in PBS. 1×10⁶ cells per transfection were spun down at 200 rcf for 3 min and resuspended in Neon Buffer R at a final concentration of 2×10⁷ cells/ml. 50 μL of cell suspension were mixed with 50 Neon Buffer R containing 10 μg of total DNA per transfection. Nucleofection used Neon 100 μL Tips with two 20 ms pulses at 1100 V. Transfected cells were transferred into plates coated with rhLaminin-521 (Gibco A29249) prefilled with StemFlex medium supplemented with 1% RevitaCell. PIGA deletion was performed with 5 μg of each pCas9 plasmid expressing gRNAs hPIGA-g1 and hPIGA-g2 and cells were selected with 200 pM proaerolysin for 1-2 weeks post-transfection. These ΔPIGA cells were used for subsequent LP-PIGA integrations. All LP integrations at HPRT1 were performed using 5 of the pLP and 2.5 μg of each pCas9 plasmid expressing HPRT1-g1 and HPRT1-g2 gRNAs, and cells were selected using a combination of 1 μg/ml puromycin and 6-TG, as indicated. H1 PL1 integrations were performed using 5 μg pPL1. Cells were treated with 200 nM 4-Hydroxytamoxifen (Tam) the day following transfection for 3 hours, selected with 5 μg/ml Blasticidin S for 8 days followed by 4 days of selection with 100 nM GCV to eliminate TK-expressing cells.

LP integrations and genomic deletions in BL6xCAST mESCs were performed using the Neon Transfection System. Cells were washed with PBS, dissociated into a single-cell suspension using TrypLE-Select (Gibco), which was neutralized with mESC medium, spun down at 200 rcf for 3 min, supernatant aspirated and cells resuspended in PBS. 1×10⁶ cells per transfection were spun down at 200 rcf for 3 min and resuspended in Neon Buffer R at a final concentration of 2×10⁷ cells/ml. Per transfection, 50 μL of cell suspension were mixed with 50 μL Neon Buffer R containing 10 μg of total DNA and nucleofected using Neon 100 μL Tips with two 20 ms pulses at 1200 V. Transfected cells were transferred into gelatin-coated plates prefilled with 80/20 medium. Piga deletion was performed with 5 μg of each pCas9 plasmid expressing gRNAs mPiga-g1 and mPiga-g2 and cells were selected with 2 nM proaerolysin approximately 1 week post-transfection. ΔPiga cells were used for subsequent LP integrations. LP-PIGA integrations at Sox2 were performed using 5 μg of the pLP and 2.5 μg of each pCas9 plasmid expressing Sox2-g1 and Sox2-g2 gRNAs, and cells were selected with 1 μg/ml puromycin. LP-PIGA2 integration at Igf2/H19 was performed using 5 μg of the pLP-PIGA2 and 2.5 μg of each pCas9 plasmid expressing Igf2/H19-g1 and Igf2/H19-g2 gRNAs, and cells were selected with 1 μg/ml puromycin followed by selection with 1 μM GCV.

Payload deliveries in BL6xCAST mESCs were performed using a Nucleofector 2b (Lonza). Cells were washed with PBS, dissociated into a single-cell suspension using TrypLE-Select, which was neutralized with mESC medium, spun down at 200 rcf for 3 min, supernatant aspirated and cells resuspended in ice-cold PBS, counted, and 5×10⁶ cells per transfection were spun down at 200 rcf for 3 min and resuspended in a room temperature mixture of 82 μL Nucleofector Solution and 18 μL Nucleofector Supplement from the Mouse ES Cell Nucleofector kit (Lonza VPH-1001). Per transfection, 100 μL of cell suspension were mixed with 10 μL TE containing 2.25-5 μs of total DNA, and nucleofected using program A-23. PL1 deliveries were performed with 1.5 μg pPL1-BBTK and 0.75 μg pCAG-Cre (Addgene plasmid #13775). pSox246 kb-MC deliveries (failed deliveries) were performed with 35 μg pSox246 kb-MC. Payload-transfected mESCs were treated with 200 nM Tam for 4 hours before and 24 hours after transfection. Cells were selected with blasticidin constitutively starting day 1 post-transfection and with 2 nM proaerolysin for 2 days starting day 14 post-transfection. pSox246 kb-MC-BBTK deliveries were performed with 3 μg pSox246 kb-MC-BBTK and 1 μg pCAG-Cre. Payload-transfected mESCs were treated with 200 nM Tam for 24 hours before and after transfection. mESCs were grown for 10 days with blasticidin. On days 11 and 12, 1 nM proaerolysin was added, and on days 13 and 14, 1 μM GCV was also added. pSox2143 kb delivery was performed with 0.3 μg pSox2143 kb and 2 μg pCAG-iCre (Addgene plasmid #89573). Payload-transfected mESCs were selected with blasticidin for 2 days starting day 1 post-transfection and with 2 nM proaerolysin for 2 days starting day 7 post-transfection. Payload deliveries to BL6xCAST Igf2/H19 were performed with 5 pSox246 kb-MC-BBTK or pSox246 kb and 2 μg pCAG-iCre. Cells were selected with blasticidin either transiently during days 1 and 2 post transfection (pSox246 kb) or constitutively (pSox246 kb-MC-BBTK), followed by 2 nM proaerolysin selection during days 7 and 8 post-transfection. pSox2^{46kb}-MC-BBTK transfected cells were further selected with 1 μM GCV during days 9 and 10 post-transfection.

### PCR Genotyping

Genomic DNA was extracted either using the DNeasy Blood & Tissue kit (QIAGEN 69506), according to the manufacturer's protocol, or by a crude extraction protocol, applied when a large number of samples were processed. For crude DNA extraction, cells were grown to confluency in 96-well plates and washed with PBS. After removing the PBS, plates were frozen at −80° C. for at least 30 min and then thawed at room temperature. Cells were resuspended in 100 μL/well TE buffer (pH 8.0) supplemented with 0.3 mg/ml proteinase K (Thermo Scientific E00491). Mixtures were triturated several times to ensure lysis. Lysates were transferred to PCR plates and plates were sealed, spun down, and incubated at 37° C. for 1 hour and 99° C. for 10 min. Plates were spun down and left to cool down at room temperature. Typical concentrations obtained from 80% confluent wells of mESCs were 100-300 ng/μL, according to Nanodrop measurements.

PCR was conducted with 50-100 ng column-prepped DNA or with 1-2 μL of crude extract using either 2×GoTaq Green Mastermix (Promega PRM7123) or Phusion Hot Start Flex 2×Master mix (New England Biolabs M0536L) according to the manufacturers' protocols. Genotyping primers are listed in Supplementary Table 5. 8-10 μL of amplified PCR products were separated on a 1-2% agarose gel and visualized with ethidium bromide on a BIO-RAD Gel Doc XR+System. Image color was inverted.

Quantitative Real-Time PCR (qRT-PCR)

Total RNA was extracted using RNeasy Mini kit (QIAGEN) and 1-2 μg were reverse-transcribed using the High Capacity Reverse Transcription Kit (Life Technologies 4368814) according to the manufacturer's protocol. Quantitative Real-Time PCR (qRT-PCR) was performed using the KAPA SYBR FAST (Kapa Biosystems KK4610) on a LightCycler480 Real-Time PCR System (Roche). Expression was calculated using the ΔCt method. Relative Expression was calculated by dividing the average level of each gene to that of the housekeeping gene GAPDH/Gapdh measured in the same cDNA sample. qRT-PCR primers and annealing temperatures are listed in Supplementary Table 6. When data are displayed as bar charts, error bars represent standard deviations of technical replicates.

### Cell Staining and Flow Cytometry

Crystal Violet (CV) staining was performed by incubating plates for 5 min with CV solution (10 mM CV, 10% EtOH in water), followed by 3-5 gentle washes with water. PrestoBlue (ThermoFisher Scientific A13262) staining was performed according to the manufacturer's protocol. For CD59/HLA analysis, cells were washed with PBS, singularized using TrypLE-Select and neutralized with DMEM supplemented with 10% FBS. 1 million cells per sample were spun down at 500 rcf for 1 minute, and the supernatant was removed. Cell pellets were resuspended in staining solution containing DMEM, 10% FBS, 10% anti-CD59-FITC (BIO-RAD MCA1054F) and 10% anti HLA-PE (Invitrogen 12-9983-42) and incubated on ice in the dark for 30 min with occasional gentle mixing. Staining solution was topped with 0.5 mL ice-cold PBS, samples were spun down (500 rcf, 1 minute) and supernatants were aspirated. This washing step was repeated once more, and samples were resuspended in 0.3 mL ice-cold PBS, filtered and placed on ice until analysis. Flow cytometry was performed on a BD Accuri C6 instrument and results were analyzed using the FlowJo software.

### Lentiviral Infection and Cre Reporter Assay

For production of lentiviral particles, 1×10⁷ HEK-293T cells were resuspended in growth media (as described above) and transfected with 20 μg lentiviral vector, 20 μg psPAX2 packaging plasmid and 10 μg pMD2.G envelope plasmid using the Calcium Phosphate method. Cells were then plated in a 10 cm dish and cultured for one day. On the second day, media was refreshed and cells were incubated at 32° C. Viral supernatants were collected on the morning and evening of the third and fourth days, passed through a 0.22 μm cellulose acetate filter and concentrated approximately 25-fold using an Amicon Ultra-15 Centrifugal Filter (Millipore UFC903024). Cells were infected with concentrated virus, diluted in appropriate media in the presence of 8 μg/ml polybrene (Sigma TR1003G) for approximately 16 hours at 37° C. One or more days following infection, cells harboring Cre^{ERT2} were treated with 4-Hydroxytamoxifen (tamoxifen, Sigma T176) and were then assayed for DsRed and GFP expression by flow cytometry on a BD Accuri C6 machine. Cre activity was calculated using the FlowJo software as the % of GFP-positive cells of the total infected (fluorescent) cells.

Preparation of Illumina dsDNA Libraries

Genomic DNA was isolated from cells using the DNeasy Blood and Tissue Kit (Qiagen) according to the manufacturer's protocol. 1000 ng of DNA was sheared to approximately 500-900 bp in a 96-well microplate using the Covaris LE220 (450 W, 10% Duty Factor, 200 cycles per burst, and 90 second treatment time). Sheared DNA was purified using the DNA Clean and Concentrate-5 Kit (Zymo Research), and the concentration was measured on a Nanodrop instrument (Invitrogen). DNA fragments were end-repaired with T4 DNA polymerase, Klenow DNA polymerase, and T4 polynucleotide kinase (NEB), and A-tailed using Klenow (3′-5′ exo-, NEB). Illumina-compatible adapters were subsequently ligated to DNA ends, and DNA libraries were amplified with KAPA 2×Hi-Fi Hotstart Readymix (Roche).

### Targeted Resequencing Using Capture-Seq

Baits for sequence capture were prepared from BAC or plasmid DNA containing the sequence of interest. Biotin-16-dUTP (Roche) was incorporated into bait DNA using a Nick Translation kit (Roche). The reaction (total volume 20 μL) was set-up in a 200 PCR tube on ice as follows: 2 μg of BAC DNA, 10 μL of 0.1 mM Biotin-dUTP/dNTP mixture (1 volume Biotin-16-dUTP, 2 volumes dTTP, 3 volumes dATP, 3 volumes dCTP and 3 volumes dGTP), 2 μL of 10×nick translation buffer and 2 μL of enzyme mixture. Nick translation was carried out at 15° C. for 16 hours or 8 hours (for BAC or plasmid DNA, respectively) in a thermal cycler. The reaction was stopped by addition of 1 μL 0.5 M EDTA and heating at 65° C. for 10 min or cooling at 4° C. overnight. Biotinylated baits were purified by ethanol precipitation, resuspended in 50 mL H₂O, and the concentration was measured on a Nanodrop instrument. Baits were stored at −20° C.

Targeted sequencing using in-solution hybridization capture (Capture-Seq) was performed as described previously⁵⁰ with modifications. 1 μg biotinylated DNA bait and μg Cot-1 human or mouse DNA (Invitrogen) were combined with universal and sample-specific blocking oligos and lyophilized using a SpeedVac. Lyophilized DNA was resuspended in 12 μL TE (pH 7.5) and overlaid with mineral oil. In a thermal cycler, the DNA mixture was denatured at 96° C. for 5 min, incubated at 65° C. for an additional min, and then 12 μL of 2×hybridization buffer (1.5 M NaCl, 40 mM sodium phosphate buffer (pH 7.2), 10 mM EDTA (pH 8), 10×Denhardt's and 0.2% SDS) was added to the DNA, and the mixture was pre-hybridized for 6 hours at 65° C.

A total of 1 μg from up to 2-8 libraries were pooled into a single 200 μL PCR tube for a single capture reaction. Library DNA was diluted in H₂O to a final volume of 12 μL and overlaid with mineral oil. Library DNA was denatured at 96° C. for 5 min, incubated at 65° C. for an additional 15 min, and then 12 μL of 2×hybridization buffer was added to the denatured DNA library. The entire volume (24 μL) of denatured library DNA was added to the tube of pre-hybridized bait DNA, and the mixture was incubated at 65° C. for 16-22 hours. For each capture reaction, 50 μL of MyOne streptavidin-coated magnetic beads (Invitrogen) were washed with 1×B&W buffer (5 mM Tris-HCl pH 7.5, mM EDTA, 1 M NaCl) three times, and then resuspended in 150 μL 1×B&W buffer in a low-retention microcentrifuge tube. The hybridization mix (48 μL) plus 48 μL 2×B&W buffer (10 mM Tris-HCl pH 7.5, 1 mM EDTA, 2 M NaCl) were then combined with the pre-washed magnetic beads, and incubated at room temperature for 30 min with rotation. The magnetic beads were washed once at 25° C. for 15 min in 1×SSC with SDS and three times at 65° C. for 15 min in 0.1×SSC with 0.1% SDS. To denature the captured library DNA, the beads were resuspended in 100 μL 100 mM NaOH, and incubated at room temperature for 10 min. After allowing the beads to separate on a magnetic rack, the supernatant (containing enriched library DNA) was transferred to a new tube, neutralized with 100 μL 1 M Tris-HCl pH 7.5, and purified using the DNA Clean and Concentrate-5 Kit (Zymo Research). Four microliters of the captured library DNA were evaluated using qPCR to determine the optimal number of final PCR amplification cycles. Captured libraries were then amplified with KAPA Hi-Fi Hotstart Readymix (Roche).

### Sequencing Processing

Illumina libraries were sequenced in paired-end mode on an Illumina NextSeq 500 operated at the Institute for Systems Genetics or a NovaSeq 6000 operated by the NYU Langone Health Genome Technology Center. Reads were demultiplexed with Illumina bcl2fastq v2.20 requiring a perfect match to indexing BC sequences. All whole-genome sequencing and Capture-Seq data were processed using a uniform mapping and peak calling pipeline. Illumina sequencing adapters were trimmed with Trimmomatic v0.39⁵¹. Sequencing reads were aligned using BWA v0.7.17⁵² to a genome reference (GRCh38/hg38 or GRCm38/mm10) including unscaffolded contigs and alternate references, as well as independently to custom references for relevant vectors. PCR duplicates were marked using samblaster v0.1.24⁵³. Generation of per-base coverage depth tracks and quantification was performed using BEDOPS v2.4.35⁵⁴. Data were visualized using the UC SC Genome Browser.

### Genotype Analysis

Variant calling was performed on sequenced BL6xCAST samples to verify correct allele-specific engineering using a standard pipeline based on bcftools v1.9:
bcftools mpileup--redo-BAQ--adjust-MQ 50--gap-frac 0.05--max-depth 10000--max-idepth 200000-a DP,AD--output-type u|
bcftools call--keep-alts-ploidy 1--multiallelic-caller-f GQ--output-type u

Raw Pileups were Filtered Using:
bcftools norm--check-ref w--output-type u|
bcftools filter-i “INFO/DP>=10 & QUAL>=10 & GQ>=99 & FORMAT/DP>=10”--SnpGap 3--IndelGap 10--set-GTs.--output-type u|
bcftools view-i ‘GT=“alt”’--trim-alt-alleles--output-type z

SNVs called in each sample were intersected with expected BL6/CAST heterozygous sites based on known variants called for CAST/EiJ 55.

### Analysis of Integration Junctions Using Bamintersect

Bamintersect enables efficient filtering and analysis of paired-end sequencing reads mapped independently to two different reference sequences, typically a mammalian reference genome (hg38 or mm10) and an engineered reference of interest (typically a LP or payload). To identify junctions between the two references in an unbiased fashion, baminter sect searches for read pairs where each read is mapped to a different genome. For LP/PL genomes, the read's mate is required to be unmapped to that genome. Reads must be fully mapped with <1 mismatched bases and no clipping, insertions, or deletions, and duplicate or supplementary alignments are excluded. Bamintersect filters reads (minimum of 20 bp mapping outside) against satellite repeats as well as uninformative regions defined as sequences of >120 bps with >85% similarity for the following contexts: for LP integrations, genomic regions corresponding to LP components hmPIGA, human EIF1 poly(A), ERT2 (ESR1), pEF1α (EEF1A1) and the homology arms; for payload integrations, the LP/payload shared regions pEF1α and lox sites, and the deleted genomic region; for pCas9, the human U6 promoter and hmPIGA.

Informative reads with the same strand and mapping to within 500 bp of each other were clustered for reporting. Regions below 75 bp or with fewer than 1 read/10M reads sequenced were excluded.

SUPPLEMENTARY TABLE 1
Cloning primers
Primers used for cloning plasmids.

| SEQ ID NO. | Primer Name | Primer sequence (5′ to 3′) |
|---|---|---|
| 2 | oJML0053 | TTTCCATAGGCTCCGCCCCC |
| | | CTGAC |
| 3 | OJML0056 | GAGAGCAATCCCGCAATCTT |
| | | CAGTGGTGTG |
| 4 | oJML0057 | CACACCACTGAAGATTGCGG |
| | | GATTGCTCTC |
| 5 | oJML0058 | TGATTACTATTAATAACTAG |
| | | TCAATAATCAATGTCAACGC |
| | | GGTATTTCACACCGCATAGA |
| 6 | oJML0067 | GCAATGATACCGCGAGATCC |
| | | ACGCTCACCGGCTCCAGATT |
| 7 | oJML0068 | AATCTGGAGCCGGTGAGCGT |
| | | GGATCTCGCGGTATCATTGC |
| 8 | oJML0069 | TTATTTTTATAGCACGTGAT |
| | | GAAAAGGACCAACACAGTCC |
| | | TTTCCCGCAATTTTC |
| 9 | oJML0070 | AAAAAGAAAATTGCGGGAAA |
| | | GGACTGTGTTGGTCCTTTTC |
| | | ATCACGTGCTATAAAAATAA |
| 10 | oJML0126 | GTGGTGGAAGACTCGAGCAT |
| | | GACCGAGTACAAGCCCAC |
| 11 | oJML0129 | GTTGGTGGCGCCGCTGCCGT |
| | | TAGCCTCCCCCATCTCCC |
| 12 | oJML0130 | TGAAGCAGGCCGGCGACGTG |
| | | GAGGAGAACCCCGGCCCCAT |
| | | GGC |
| | | CAATTTACTGACCGTAC |
| 13 | oJML0131 | GGAGCGCCAGACGAGGCCAA |
| | | TCATCAGGATC |
| 14 | oJML0132 | GATCCTGATGATTGGCCTCG |
| | | TCTGGCGCTCC |
| 15 | oJML0133 | TGCGATGAAGTAGAGCCCGC |
| | | AGTGGCCAAGTGGCTTTGGT |
| | | CCGT |
| | | TTCCTCCACGGATGCC |
| 16 | oJML0134 | GAAACCCTCTGCCTCCCCCG |
| | | TGATGTAATACTTTTGCAAG |
| | | GAAT |
| | | GCGATGAAGTAGAGCC |
| 17 | oJML0135 | GGGAGGCAGAGGGTTTCCCT |
| | | GCCACAGCTTGATGAAGATG |
| | | AGGCCAACCTTCTATCAGAG |
| 18 | oJML0136 | GTGGTGGAAGACTCTGAATG |
| | | TCTCAAAAAACAAACGAACA |
| | | AAAAACCAG |
| 19 | OJML0137 | CATGACCCGCAAGCCCGGTG |
| | | CCATGCCCACGCTACTGCGG |
| 20 | oJML0138 | GGGGTTCTCCTCCACGTCGC |
| | | CGGCCTGCTTCAGCAGGCTG |
| | | AAGTTGGTGGCGCCGCTGCC |
| 21 | oJML0139 | GGCGCCACCAACTTCAGCCT |
| | | GCTGAAGCAGGCCGGCGACG |
| | | TGGAGGAGAACCCCGGCCCC |
| 22 | oJML0144 | AAACCCGCAGTAGCGTGGGC |
| | | ATGGCACCGGGCTTGCGGGT |
| 23 | oJML0145 | GTGGTGCGTCTCTTCGGGGC |
| | | TCCGGTGCCCGTCAGTG |
| 24 | OJML0146 | CACCACCGTCTCGGCTCTCA |
| | | CGACACCTGAAATGGAAG |
| 25 | oRB_036 | ATCGGCGGCCGCTGGAATTA |
| | | TAACTTCGTATAGC |
| 26 | oRB_037 | GGCCATCGATTTACTTGTAC |
| | | AGCTCGTCC |
| 27 | oRB_061 | GATTATTAGGGATAACAGGG |
| | | TAATATAACTTCGTATAGGA |
| | | TACT |
| | | TTATACGAAGTTATGGCTCC |
| | | GGTGCC |
| 28 | oRB_062 | GCATGGACGAGCTGTACAAG |
| | | GGCAGTGGAGAGGGCAGAG |
| 29 | oRB_063 | CCTCTGCCCTCTCCACTGCC |
| | | CTTGTACAGCTCG |
| 30 | oRB_064 | CCTAAAATTACCCTGTTATC |
| | | CCTAATAACTTCGTATAATG |
| | | TATGC |
| | | TATACGAAGTTATTCCCCAG |
| | | CATGCCTGCTATT |

SUPPLEMENTARY TABLE 2
Homology arm cloning primers
Primer pairs used to clone left and right
homology arms (HAs) using BsaI-
mediated Golden Gate reactions
(BsaI sites are in lower case). gRNA binding sites
(underlined) and PAMs (bold) were encoded externally
in the primer sequences were indicated.

| SEQ | | HA | | | Forward primer | Reverse primer |
|---|---|---|---|---|---|---|
| ID | Species/ | Length | gRNA | | sequence | sequence |
| NO. | Locus | (kbps) | sites | HA | (5′ to 3′) | (5′ to 3′ |
| 31/ | Human/ | 1 | Yes | Left | TGTGTggtctcACCCTTTCATAC | GTGGTGggtctcACGTTGGC |
| 32 | HPRT1 | | | | CCATGTAAGGTTGAGGTGAA | GCGCGTTGCTTCATG |
| | | | | | GAGACTGAGGTCCAGAG | |
| 33/ | | | | Right | GTGGTGggtctcGTATGTTGAGG | GTGGTGggtctcAGGATAT |
| 34 | | | | | TAGATGTTACCACATGT | GAAGCAACGCGCGCCGGTA |
| | | | | | | GGTCTGGACCTGCACTTC |
| | | | | | | TTCA |
| 35/ | Human/ | 1 | No | Left | GTGTGTggtctcACCCTTGAAGA | GTGGTGggtctcACGTTGGC |
| 36 | HPRT1 | | | | GACTGAGGTCCAGAG | GCGCGTTGCTTCATG |
| 37/ | | | | Right | GTGGTGggtctcGTATGTTGAGG | GTGGTGggtctcAGGATTCT |
| 38 | | | | | TAGATGTTACCACATGT | GGACCTGCACTTCTTCA |
| 39/ | Human/ | 0.25 | No | Left | GTGTggtctcACCCTGTACAAAA | GTGGTGggtctcACGTTGGC |
| 40 | HPRT1 | | | | CTACAGAGCAGTTAAGTG | GCGCGTTGCTTCATG |
| 41/ | | | | Right | GTGGTGggtctcGTATGTTGAGG | GGTGggtctcAGGATGTTAT |
| 42 | | | | | TAGATGTTACCACATGT | ACGACGCCAAACTGCC |
| 43/ | Human/ | 0.1 | No | Left | GTGTggtctcACCCTAAGGTCTT | GTGGTGggtctcACGTTGGC |
| 44 | HPRT1 | | | | GGGAATGGGACG | GCGCGTTGCTTCATG |
| 45/ | | | | Right | GTGGTGggtctcGTATGTTGAGG | GGTGggtctcAGGATGAGGG |
| 46 | | | | | TAGATGTTACCACATGT | TAGCCAAGTGGACC |
| 47/ | Mouse/ | 0.15 | Yes | Left | GTGTggtctcACCCTCAAGTCTG | GTGGTGggtctcACGTTGAA |
| 48 | Sox2 | | | | AAGTAGTTCAGGAGGGTTTG | CTACTTCAGACTTGGGC |
| | | | | | AGGCCAGGAAGGGAT | |
| 49/ | | | | Right | GTGGTGggtctcGTATGGTTCGG | GGTGggtctcAGGATGAGCT |
| 50 | | | | | GGACGGTGTTAATATTCTTC | GCAAAGGCTCCCGTTAGG |
| | | | | | | AATGAATGCGGATGCCTT |
| | | | | | | GC |
| 51/ | Mouse/ | 0.1 | Yes | Left | GGTGggtctcACCCTCATCGTGC | GGTGggtctcACGTTTTGCTA |
| 52 | Igf2/ | | | | CCATAGCAATGATGGGCCCA | TGGGCACGATGCTG |
| | H19 | | | | GGTGAAGAGTCAACC | |
| 53/ | | | | Right | GGTGggtctcGTATGAGAGCTG | GGTGggtctcAGGATGAGA |
| 54 | | | | | GGATAATCTCTTT | TTATCCCAGCTCTGGGAGG |
| | | | | | | AGCTATTTTAGAAGGACT |
| | | | | | | CCC |

SUPPLEMENTARY TABLE 3
Genomic coordinates for engineered loci.
Coordinates of deletions at engineered loci, BACs, and payload constructs.

| Species | | | |
|---|---|---|---|
| (genome) | Locus | Coordinates | Description |
| Human | PIGA | chrX: 15318514- | Deleted region in ΔPIGA hESCs |
| (hg38) | | 15336428 | |
| Human | HPRT1 | chrX: 134459947- | HPRT1 region replaced with LP-TK and LP-PIGA |
| (hg38) | | 134501642 | |
| Human | HPRT1 | chrX: 134429208- | HPRT1 BAC²⁵ used for HPRT1 bait |
| (hg38) | | 134529874 | |
| Mouse | Piga | chrX: 164418679- | Deleted region in ΔPiga mESCs |
| (mm10) | | 164435590 | |
| Mouse | Piga | chrX: 164376257- | BAC RP23-32H22 (BACPAC Resources Center) |
| (mm10) | | 164581046 | used for mouse Piga bait |
| Mouse | Sox2 | chr3: 34577661- | BAC RP23-144O8 (BACPAC Resources Center) |
| (mm10) | | 34798754 | used for Sox2 bait and for cloning Sox2 payloads |
| Mouse | Igf2/H19 | chr7: 142458937- | BAC RP23-50N22 (BACPAC Resources Center) |
| (mm10) | | 142702723 | used for Igf2/H19 bait |
| Mouse | Sox2 | chr3: 34631454- | Sox2 region replaced with LP-PIGA; Sox2^{143kb} |
| (mm10) | | 34774117 | payload |
| Mouse | Sox2 | chr3: 34631454- | Sox2^{46kb} payload |
| (mm10) | | 34677464 | |
| Mouse | Igf2/H19 | chr7: 142,520,698- | Igf2/H19 region replaced with LP-PIGA2 |
| (mm10) | | 142,520,706 | |

SUPPLEMENTARY TABLE 4
gRNAs used for landing pad insertion.
gRNAs used to cut loci termini.

| SEQ | | | | | |
|---|---|---|---|---|---|
| ID | gRNA | Species | | Genomic | |
| NO. | Name | (genome) | Sequence | coordinates | Strand |
| 55 | hPIGA- | Human | GTTATACTTT | chrX:15336426- | (−) |
| | g1 | (hg38) | GGCCAGCATG | 15336445 | |
| 56 | hPIGA- | Human | AACATCTAGC | chrX: 15318498- | (+) |
| | g2 | (hg38) | CACATCCATT | 15318517 | |
| 57 | HPRT1- | Human | ATGAAGCAAC | chrX: 134459930- | (+) |
| | g1 | (hg38) | GCGCGCCGGT | 134459949 | |
| 58 | HPRT1- | Human | TTCATACCCA | chrX: 134501626- | (+) |
| | g2 | (hg38) | TGTAAGGTTG | 134501645 | |
| 59 | mPiga- | Mouse | GGCATGCTTT | chrX: 164418663- | (+) |
| | g1 | (mm10) | GTGGTCGTTC | 164418682 | |
| 60 | mPiga- | Mouse | CCCGCGGGCA | chrX: 164435574- | (+) |
| | g2 | (mm10) | GCCTATATAA | 164435593 | |
| 61 | Sox2- | Mouse | CAAGTCTGAA | chr3: 34631437- | (+) |
| | g1 | (mm10) | GTAGTTCAGG | 34631456 | |
| 62 | Sox2- | Mouse | GAGCTGCAAA | chr3: 34774101- | (+) |
| | g2 | (mm10) | GGCTCCCGTT | 34774120 | |
| 63 | Sox2- | Mouse | CATTGGCAGT | chr3: 34677448- | (+) |
| | g3 | (mm10) | GTTGTATAGG | 34677467 | |
| 64 | Igf2/ | Mouse | CATCGTGCCC | chr7: 142520681- | (+) |
| | H19-g1 | (mm10) | ATAGCAATGA | 142520700 | |
| 65 | Igf2/ | Mouse | GAGATTATCC | chr7: 142678067- | (−) |
| | H19-g2 | (mm10) | CAGCTCTGGG | 142678086 | |

SUPPLEMENTARY TABLE 5
Genotyping primers
Primer pairs used to verify engineered cells
correspond to the listed figure and assay.

| | | | Forward | Reverse |
|---|---|---|---|---|
| SEQ | | | primer | Primer |
| ID | | | Sequence | Sequence |
| NO. | FIG. | Assay | (5' to 3') | (5' to 3') |
| 66/67 | 1 | LP-TK at | CAGGATATTT | GGGACTGTGG |
| | | HPRT1 L Jx | CTCTGTTGCC | GCGATGTG |
| | | | CA | |
| 68/69 | 1 | LP-TK at | ACCCACAGCT | GGTGGAATAC |
| | | HPRTI R Jx | TCTCAACGG | AACTGCCTGG |
| 70/71 | 1 | LP-PIGA at | GACTGGGGCT | GGGACTGTGG |
| | | Sox2 L Jx | TCTCAGAGTT | GCGATGTG |
| | | | C | |
| 72/73 | 1 | LP-PIGA at | ACCCACAGCT | AGTGACTGCA |
| | | Sox2 R Jx | TCTCAACGG | GCAGACTTGG |
| 74/75 | 1 | Ori | TTTCCATAGG | GTTACCGGAT |
| | | | CTCCGCCCCC | AAGGCGCAGC |
| | | | CTGAC | |
| 76/77 | 1 | Sox2[B16]- | GACTGGGGCT | GATCTCTGGT |
| | | 5' | TCTCAGAGTT | GTACCAGTGT |
| | | | C | GTCC |
| 78/79 | 3 | PL1 at | CCTGATCTGG | AGAGGTTCAG |
| | | HPRT1 | GTGACTCTAG | CAGTGGGAAG |
| | | | G | |
| 80/81 | 4 | PL1 at | AGAATAGCAG | AGTGACTGCA |
| | | Sox2 R Jx | GCATGCTGGG | GCAGACTTGG |
| 82/83 | 4 | MC L Jx | GAGGCAGGGC | AGAATAGCAG |
| | | | AATCAGAAGT | GCATGCTGGG |
| 84/85 | 4 | Sox2^{46kb} at | GGGAGGGGGC | AGTGACTGCA |
| | | Sox2 R Jx | CCTGCCG | GCAGACTTGG |
| 86/87 | 4 | Sox2^{143kb} at | GACTGGGGCT | GGGACTGTGG |
| | | Sox2 L Jx | TCTCAGAGTT | GCGATGTG |
| | | | C | |
| 88/89 | 4 | Sox2^{143kb} at | AGACTTGTTT | GCCACTGAGA |
| | | Sox2 R Jx | TCCTCCTGCC | CCGAGGT |
| | | | T | |
| 90/91 | 4 | LP-PIGA at | ACCCACAGCT | AGTGACTGCA |
| | | Sox2 R Jx | TCTCAACGG | GCAGACTTGG |
| 92/93 | 8 | WT PIGA | TTACTATCTG | CCATGCGTCA |
| | | | GCAGGGAAGG | CAGCTGGTAC |
| | | | C | |
| 94/95 | 8 | ΔPIGA | TTACTATCTG | TGTGATGGGC |
| | | | GCAGGGAAGG | ATAAAAGGCT |
| | | | C | ACT |
| 96/97 | 8 | WT HPRT1 | TTCCCAGCAA | ACAAGGCCAA |
| | | | CAAAGTAGGA | CAGCAGTCTG |
| | | | G | |
| 98/99 | 8 | LP-PIGA at | TGGTGCGATC | TTACGTCTGC |
| | | HPRT1 L Jx | TCAGCTCAGT | TGCAGGCGCG |
| 100/101 | 8 | LP-PIGA at | ACCCACAGCT | ACAAGGCCAA |
| | | HPRT1 R Jx | TCTCAACGG | CAGCAGTCTG |
| 102/103 | 11 | LP Jx | ATGAGAACCC | CCAACTTCTC |
| | | | GGAAAGAGGG | GGGGACTGTG |
| 104/105 | 11 | L Jx 1 | ATGAGAACCC | AGAATAGCAG |
| | | | GGAAAGAGGG | GCATGCTGGG |
| 106/107 | 11 | L Jx 2 | ATGAGAACCC | GATCTCTGGT |
| | | | GGAAAGAGGG | GTACCAGTGT |
| | | | | GTCC |
| 108/109 | 11 | R Jx | GGGAGGGGGC | GAGGTCTTTG |
| | | | CCTGCCG | GAAGGCATGG |
| 110/111 | 12 | duplication | CCACTCCTGC | CATCCAAAGG |
| | | | CACTTCAGAG | GTGCAAAGGC |
| 112/113 | 12 | deletion | GGAAAGGCGG | CGACGTTTGA |
| | | | CAACTGTTTT | ACGGGTTTCC |

SUPPLEMENTARY TABLE 6
Quantitative PCR primers
Primer pairs used for qPCR and qRT-PCR.
Corresponding melting temperature
(Tm) is indicated.

| SEQ | | Forward primer | Reverse primer | |
|---|---|---|---|---|
| ID | | sequence | sequence | |
| NO. | Assay | (5′ to 3′) | (5′ to ′3) | Tm |
| 114/ | CreERT2 | CGATTGATTTACG | GCCATCTTCCAG | 60° C. |
| 115 | FIG. 6b) | GCGCTAAGGAT | CAGGCGCAC | |
| 116/ | CreERT2 | AATGGTTTCCCGC | AGCAATCCCCAG | 60° C. |
| 117 | (FIG. 9b) | AGAACCTGAAGA | AAATGCCAG | |
| 118/ | Human | GACCAGTCAACAG | CCTGACCAAGGA | 60° C. |
| 119 | HPRT1 | GGGACAT | AAGCAAAG | |
| 120/ | Human | GCCTGATTGAAAG | GACTGGTTGTACA | 60° C. |
| 121 | mini | AGGGCATAAGGT | TGACTTTCAGAGG | |
| | PIGA | TATAATTG | | |
| | (hmPIGA) | | | |
| 122/ | Human | TTTGCTGATGTC | CCAAAAGACGCAC | 60° C. |
| 123 | total | AGCTCGGT | CCTGTCA | |
| | PIGA | | | |
| 124/ | Mouse | AAGCCTAAGATGA | GGACGCAGCAACT | 60° C. |
| 125 | Hprt1 | GCGCAAG | GACATT | |
| 126/ | Mouse Sox2 | AACCGATGCACCGC | GAGCATTATCAGA | 57° C. |
| 127 | [BL6] | | TTTTTCC | |
| 128/ | Mouse Sox2 | AGCCGATGCACCGA | TGAGCATTATCAG | 57° C. |
| 129 | [Cast] | | ATTTTTCT | |
| 130/ | BSD | TCGCGACGATAC | GGACCTTGTGCAG | 60° C. |
| 131 | | AAGTCAGG | AACTCGT | |

The following reference listing is not an indication that any particular reference(s) is material to patentability.
1 Maurano, M. T. et al. Systematic localization of common disease-associated variation in regulatory DNA. Science 337, 1190-1195, doi:10.1126/science.1222794 (2012).
2 Palmiter, R. D. & Brinster, R. L. Germ-line transformation of mice. Annual review of genetics 20, 465-499, doi:10.1146/annurev.ge.20.120186.002341 (1986).
3 Aguet, F. et al. Genetic effects on gene expression across human tissues. Nature 550, 204-213, doi:10.1038/nature24277 (2017).
4 Maurano, M. T. et al. Identification of cellular context sensitive regulatory variation in mouse genomes. bioRxiv, 2020.2006.2027.175422, doi:10.1101/2020.06.27.175422 (2020).
Smithies, O., Gregg, R. G., Boggs, S. S., Koralewski, M. A. & Kucherlapati, R. S. Insertion of DNA sequences into the human chromosomal beta-globin locus by homologous recombination. Nature 317, 230-234, doi:10.1038/317230a0 (1985).
6 Thomas, K. R., Folger, K. R. & Capecchi, M. R. High frequency targeting of genes to specific sites in the mammalian genome. Cell 44, 419-428, doi:10.1016/0092-8674(86)90463-0 (1986).
7 Urnov, F. D. Genome Editing B.C. (Before CRISPR): Lasting Lessons from the “Old Testament”. The CRISPR journal 1, 34-46, doi:10.1089/crispr.2018.29007.fyu (2018).
8 Vierstra, J. et al. Functional footprinting of regulatory DNA. Nat Methods 12, 927-930, doi:10.1038/nmeth.3554 (2015).
9 Sanjana, N. E. et al. High-resolution interrogation of functional elements in the noncoding genome. Science 353, 1545-1549, doi:10.1126/science.aaf7613 (2016).
10 Osterwalder, M. et al. Enhancer redundancy provides phenotypic robustness in mammalian development. Nature 554, 239-243, doi:10.1038/nature25461 (2018).
11 Despang, A. et al. Functional dissection of the Sox9-Kcnj2 locus identifies nonessential and instructive roles of TAD architecture. Nat Genet 51, 1263-1271, doi:10.1038/s41588-019-0466-z (2019).
12 Boroviak, K., Fu, B., Yang, F., Doe, B. & Bradley, A. Revealing hidden complexities of genomic rearrangements generated with Cas9. Sci Rep 7, 12867, doi:10.1038/s41598-017-12740-6 (2017).
13 Richardson, S. M. et al. Design of a synthetic yeast genome. Science 355, 1040-1044, doi:10.1126/science.aaf4557 (2017).
14 Zhang, W., Mitchell, L. A., Bader, J. S. & Boeke, J. D. Synthetic Genomes. Annu Rev Biochem 89, 77-101, doi:10.1146/annurev-biochem-013118-110704 (2020).
Heintz, N. BAC to the future: the use of bac transgenic mice for neuroscience research. Nat Rev Neurosci 2, 861-870, doi:10.1038/35104049 (2001).
16 Peterson, K. R. et al. Transgenic mice containing a 248-kb yeast artificial chromosome carrying the human beta-globin locus display proper developmental control of human globin genes. Proc Natl Acad Sci USA 90, 7593-7597, doi:10.1073/pnas.90.16.7593 (1993).
17 Schedl, A., Montoliu, L., Kelsey, G. & Schutz, G. A yeast artificial chromosome covering the tyrosinase gene confers copy number-dependent expression in transgenic mice. Nature 362, 258-261, doi:10.1038/362258a0 (1993).
18 Peterson, K. R. et al. Use of yeast artificial chromosomes (YACs) in studies of mammalian development: production of beta-globin locus YAC mice carrying human globin developmental mutants. Proc Natl Acad Sci USA 92, 5655-5659, doi:10.1073/pnas.92.12.5655 (1995).
19 Seibler, J., Schubeler, D., Fiering, S., Groudine, M. & Bode, J. DNA cassette exchange in ES cells mediated by Flp recombinase: an efficient strategy for repeated modification of tagged loci by marker-free constructs. Biochemistry 37, 6229-6234, doi:10.1021/bi980288t (1998).
20 Bouhassira, E. E., Westerman, K. & Leboulch, P. Transcriptional behavior of LCR enhancer elements integrated at the same chromosomal locus by recombinase-mediated cassette exchange. Blood 90, 3332-3344 (1997).
21 Iacovino, M. et al. Inducible cassette exchange: a rapid and efficient system enabling conditional gene expression in embryonic stem and primary cells. Stem Cells 29, 1580-1588, doi:10.1002/stem.715 (2011).
22 Zhu, F. et al. DICE, an efficient system for iterative genomic editing in human pluripotent stem cells. Nucleic Acids Res 42, e34, doi:10.1093/nar/gkt1290 (2014).
23 Matreyek, K. A., Stephany, J. J. & Fowler, D. M. A platform for functional assessment of large variant libraries in mammalian cells. Nucleic Acids Res 45, e102, doi:10.1093/nar/gkx183 (2017).
24 Wallace, H. A. et al. Manipulating the mouse genome to engineer precise functional syntenic replacements with human sequence. Cell 128, 197-209, doi:10.1016/j.cell.2006.11.044 (2007).
25 Mitchell, L. A. et al. De novo assembly, delivery and expression of a 101 kb human gene in mouse cells. bioRxiv, 423426, doi:10.1101/423426 (2019).
26 St Clair, M. H., Lambe, C. U. & Furman, P. A. Inhibition by ganciclovir of cell growth and DNA synthesis of cells biochemically transformed with herpesvirus genetic information. Antimicrob Agents Chemother 31, 844-849, doi:10.1128/aac.31.6.844 (1987).
27 Friedel, R. H., Wurst, W., Wefers, B. & Kuhn, R. Generating conditional knockout mice. Methods Mol Blot 693, 205-231, doi:10.1007/978-1-60761-974-1_12 (2011).
28 Ryan, M. D., King, A. M. & Thomas, G. P. Cleavage of foot-and-mouth disease virus polyprotein is mediated by residues located within a 19 amino acid sequence. J Gen Virol 72 (Pt 11), 2727-2732, doi:10.1099/0022-1317-72-11-2727 (1991).
29 Caskey, C. T. & Kruh, G. D. The HPRT locus. Cell 16, 1-9, doi:10.1016/0092-8674(79)90182-x (1979).
30 Ran, F. A. et al. Genome engineering using the CRISPR-Cas9 system. Nat Protoc 8, 2281-2308, doi:10.1038/nprot.2013.143 (2013).
31 Yao, X. et al. Tild-CRISPR Allows for Efficient and Precise Gene Knockin in Mouse and Human Cells. Dev Cell 45, 526-536 e525, doi:10.1016/j.devce1.2018.04.021 (2018).
32 Eckersley-Maslin, M. A. et al. Random monoallelic gene expression increases upon embryonic stem cell differentiation. Dev Cell 28, 351-365, doi:10.1016/j.devce1.2014.01.017 (2014).
33 Keane, T. M. et al. Mouse genomic variation and its effect on phenotypes and gene regulation. Nature 477, 289-294, doi:10.1038/nature10413 (2011).
34 Avilion, A. A. et al. Multipotent cell lineages in early mouse development depend on SOX2 function. Genes Dev 17, 126-140, doi:10.1101/gad.224503 (2003).
35 Zhou, H. Y. et al. A Sox2 distal enhancer cluster regulates embryonic stem cell differentiation potential. Genes Dev 28, 2699-2711, doi:10.1101/gad.248526.114 (2014).
36 Li, Y. et al. CRISPR Reveals a Distal Super-Enhancer Required for Sox2 Expression in Mouse Embryonic Stem Cells. PLoS ONE 9, e114485, doi:10.1371/journal.pone.0114485 (2014).
37 Bindels, D. S. et al. mScarlet: a bright monomeric red fluorescent protein for cellular imaging. Nat Methods 14, 53-56, doi:10.1038/nmeth.4074 (2017).
38 Fillat, C., Carrio, M., Cascante, A. & Sangro, B. Suicide gene therapy mediated by the Herpes Simplex virus thymidine kinase gene/Ganciclovir system: fifteen years of application. Curr Gene Ther 3, 13-26, doi:10.2174/1566523033347426 (2003).
39 Elshami, A. A. et al. Gap junctions play a role in the ‘bystander effect’ of the herpes simplex virus thymidine kinase/ganciclovir system in vitro. Gene Ther 3, 85-92 (1996).
40 Mesnil, M., Piccoli, C., Tiraby, G., Willecke, K. & Yamasaki, H. Bystander killing of cancer cells by herpes simplex virus thymidine kinase gene is mediated by connexins. Proc Natl Acad Sci USA 93, 1831-1835, doi:10.1073/pnas.93.5.1831 (1996).
41 Iida, Y. et al. Characterization of genomic PIG-A gene: a gene for glycosylphosphatidylinositol-anchor biosynthesis and paroxysmal nocturnal hemoglobinuria. Blood 83, 3126-3131 (1994).
42 Diep, D. B., Nelson, K. L., Raja, S. M., Pleshak, E. N. & Buckley, J. T. Glycosylphosphatidylinositol anchors of membrane glycoproteins are binding determinants for the channel-forming toxin aerolysin. J Biol Chem 273, 2355-2360, doi:10.1074/jbc.273.4.2355 (1998).
43 Araten, D. J., Nafa, K., Pakdeesuwan, K. & Luzzatto, L. Clonal populations of hematopoietic cells with paroxysmal nocturnal hemoglobinuria genotype and phenotype are present in normal individuals. Proc Natl Acad Sci USA 96, 5209-5214 (1999).
44 Li, D. Rearranging Natural and Engineered Genomes: From Mobile DNA to Designer Deletion Cell Lines PhD thesis, Johns Hopkins University, (2018).
45 Laurent, J. M. et al. Big DNA as a tool to dissect an age-related macular degeneration-associated haplotype. Precis Clin Med 2, 1-7, doi:10.1093/pcmedi/pby019 (2019).
46 Fowler, D. M. & Fields, S. Deep mutational scanning: a new style of protein science. Nat Methods 11, 801-807, doi:10.1038/nmeth.3027 (2014).
47 Lin, Q. et al. RADOM, an efficient in vivo method for assembling designed DNA fragments up to 10 kb long in Saccharomyces cerevisiae. ACS synthetic biology 4, 213-220, doi:10.1021/sb500241e (2015).
48 Koo, B. K. et al. Controlled gene expression in primary Lgr5 organoid cultures. Nat Methods 9, 81-83, doi:10.1038/nmeth.1802 (2011).
49 Perez, A. R. et al. GuideScan software for improved single and paired CRISPR guide RNA design. Nat Biotechnol 35, 347-349, doi:10.1038/nbt.3804 (2017).
50 Yigit, E. et al. High-resolution nucleosome mapping of targeted regions using BAC-based enrichment. Nucleic Acids Res 41, e87, doi:10.1093/nar/gkt081 (2013).
51 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
52 Li, H. & Durbin, R. Fast and accurate short read alignment with Burrows-Wheeler transform. Bioinformatics 25, 1754-1760, doi:10.1093/bioinformatics/btp324 (2009).
53 Faust, G. G. & Hall, I. M. SAMBLASTER: fast duplicate marking and structural variant read extraction. Bioinformatics 30, 2503-2505, doi:10.1093/bioinformatics/btu314 (2014).
54 Neph, S. et al. BEDOPS: high-performance genomic feature operations. Bioinformatics 28, 1919-1920, doi:10.1093/bioinformatics/bts277 (2012).
55 Keane, T. M. et al. Mouse genomic variation and its effect on phenotypes and gene regulation. Nature 477, 289-294, doi:10.1038/nature10413 (2011).
56 Feil, R., Wagner, J., Metzger, D. & Chambon, P. Regulation of Cre recombinase activity by mutated estrogen receptor ligand-binding domains. Biochem Biophys Res Commun 237, 752-757, doi:10.1006/bbrc.1997.7124 (1997).

1. A method for insertion of a DNA payload into a chromosomal locus in mammalian cells, the method comprising:
a. introducing into the locus a first double stranded (ds) DNA template (a landing pad “LP”) that comprises 5′ and 3′ homology arms (HAs), wherein the LP encodes a positive selection marker and a negative selection marker; and wherein the LP comprises a pair of recombinase recognition sites configured to excise a segment of the LP that comprises at least the negative selection marker,
b. selecting cells that comprise the LP using the positive selection marker to obtain an isolated population of the mammalian cells that comprise the LP;
c. introducing into the isolated population of mammalian cells of b. a second dsDNA comprising a payload sequence and a positive selection marker used to select cells that comprise the payload, wherein the positive selection marker is i) within the payload sequence in the second dsDNA and is inserted into the locus, or ii) is present on a location on the second dsDNA that is not inserted into the locus;
whereby a recombinase present in the mammalian cells that recognizes the recombinase recognition sites removes at least the segment of the LP that comprises the negative selection marker in at least some of the mammalian cells, such that at least the segment of the LP comprising the negative selection marker is replaced by the payload by homologous recombination of the payload into the locus in at least some of the mammalian cells;
d. exposing the mammalian cells of c. to an agent that acts on the negative selection marker such that only mammalian cells that contain the LP and the negative selection marker but not the payload are killed; and subsequently
e. separating the mammalian cells that comprise the payload but do not contain the LP to obtain isolated viable mammalian cells that comprise the payload.

2. The method of claim 1, wherein the LP is introduced using a nuclease system selected from an RNA-guided clustered regularly interspaced short palindromic repeats (CRISPR) enzyme, a Transcription activator-like effector nuclease (TALEN), a zinc finger nuclease, or a MAD-series nuclease.

3. The method of claim 1, wherein the mammalian cells into which the LP is introduced in a. comprise an endogenous mutated gene that encodes Phosphatidylinositol Glycan Anchor Biosynthesis Class A (PIGA) enzyme such that the function of the PIGA enzyme is reduced or eliminated relative to a non-mutated gene that encodes the PIGA enzyme, and wherein the LP comprises a sequence encoding a functional PIGA enzyme as the negative selection marker.

4. The method of claim 3, wherein the agent that acts on the negative selection marker is Proaerolysin.

5. The method of claim 1, wherein the wherein LP comprises a sequence encoding a herpes simplex virus type 1—thymidine kinase (HSV1-TK).

6. The method of claim 1, wherein the agent that acts on the negative selection marker is ganciclovir.

7. The method of any claim 1, wherein the payload is only inserted into the locus on one homologous chromosome to thereby provide a heterozygous chromosome pair in which only one chromosome in the pair comprises the payload.

8. The method of claim 1, wherein the positive selection marker is within the payload sequence in the second dsDNA and is inserted into the locus with the payload.

9. The method of claim 1, wherein the positive selection marker is present on a location on the second dsDNA that is not inserted into the locus, and wherein the payload is inserted into the locus without the positive selection marker.

10. The method of claim 1, wherein the mammalian cells are stem cells.

11. The method of claim 8, wherein the mammalian cells are stem cells.

12. The method of claim 9, wherein the mammalian cells are stem cells.

13. The method of claim 10, wherein the mammalian stem cells are embryonic stem cells.

14. A mammalian cell made according to the method of claim 1.

15. The mammalian cell of claim 14, wherein the mammalian cell is a stem cell.

16. The mammalian cell of claim 14, wherein the mammalian cell is an embryonic stem cell.

17. A non-human transgenic mammal comprising one or more mammalian cells of claim 14.

18. The non-human transgenic mammal of claim 17, wherein the non-human transgenic mammal is a mouse.
